(19) United States
(12) Patent Application Publication
(10) Pub. No.: US 2026/0216027 A1
(43) Pub. Date: Jul. 30, 2026

(54) COMPOSITION COMPRISING AT LEAST N,N-DICARBOXYMETHYLGLUTAMIC ACID, AT LEAST ONE COLOURING AGENT AND AT LEAST ONE FATTY AMINE

(71) Applicant: L'OREAL, Paris (FR)

(72) Inventor: Samira RHARBI, Saint Ouen (FR)

(21) Appl. No.: 19/143,258

(22) PCT Filed: Dec. 28, 2023
(86) PCT No.: PCT/EP2023/087942

§ 371 Date: Jun. 25, 2025

(30) Foreign Application Priority Data
Dec. 29, 2022 (FR) FR2214667

(51) Int. Cl. A61K 8/44; A61K 8/41; A61Q 5/06; A61Q 5/10

(52) U.S. Cl. CPC A61K 8/44; A61K 8/41; A61Q 5/065; A61Q 5/10; A61K 2800/4324; A61K 2800/5426

(57) ABSTRACT

The present invention relates to a cosmetic composition comprising:
a) at least one compound chosen from N,N-dicarboxymethylglutamic acid, salts thereof, solvates thereof, solvates of the salts thereof and mixtures thereof;
b) at least one colouring agent chosen from oxidation dyes, direct dyes and mixtures thereof,
c) at least one compound chosen from fatty amines, salts thereof and mixtures thereof.

The present invention relates to a composition comprising at least N,N-dicarboxymethylglutamic acid, at least one colouring agent and at least one fatty amine.

### TECHNICAL FIELD

Many people have sought for a long time to modify the colour of their hair and in particular to mask their grey hair.

Two major methods for dyeing human keratin fibres, and in particular the hair, are known.

One of these methods is oxidation dyeing or permanent dyeing. This dyeing method uses one or more oxidation dye precursors and usually one or more oxidation bases optionally combined with one or more couplers.

In general, oxidation bases are chosen from ortho- or para-phenylenediamines, ortho- or para-aminophenols and heterocyclic compounds. These oxidation bases are colourless or weakly coloured compounds which, when combined with oxidizing products, can give access to coloured species.

The shades obtained with these oxidation bases are quite often varied by combining them with one or more couplers, these couplers being notably chosen from aromatic meta-diamines, meta-aminophenols, meta-diphenols and certain heterocyclic compounds such as indole compounds.

The variety of molecules used as oxidation bases and couplers allows a wide range of colours to be obtained. This type of dyeing also makes it possible to obtain permanent colourings, but the use of oxidizing agents may lead to degradation of the keratin fibres.

The second dyeing method, known as direct dyeing or semi-permanent dyeing, comprises the application of direct dyes, which are molecules with affinity for the fibres and which colour even in the absence of an oxidizing agent added to the compositions containing them. Given the nature of the molecules used, they tend rather to remain on the surface of the fibre and penetrate relatively little into the fibre, when compared with the small molecules of oxidation dye precursors.

The direct dyes generally used are chosen from nitrobenzene, anthraquinone, nitropyridine, azo, methine, azomethine, xanthene, acridine, azine and triarylmethane direct dyes. The chemical species used may be nonionic, anionic (acidic dyes) or cationic (basic dyes). Direct dyes may also be natural dyes.

Dyeing the hair with natural direct dyes has been known since ancient times.

Compositions containing one or more natural direct dyes are applied to keratin fibres for the time required to obtain the desired colouring and are then rinsed out.

However, the resulting colourings are colourings which may be particularly chromatic but are temporary or semi-permanent since their desorption from the surface and/or core of the fibre is responsible for their lack of dyeing power and their poor resistance to washing. In addition, these compositions require relatively long leave-on times. The leave-on times may vary from several tens of minutes to several hours (overnight) depending on the desired intensity, with no ability to control the result. The result varies as a function of the fibres to be dyed and of the nature of the natural dye(s) used.

Thus, there is a real need for a composition for dyeing keratin fibres, in particular human keratin fibres such as the hair, which does not have the abovementioned drawbacks, i.e. which is capable of giving good colour build-up, intensity and chromaticity while at the same time having low (therefore good) selectivity and good persistence and which is capable of giving good dyeing performance, even after a period of storage, while at the same time having good working qualities, and good rheological properties such as in terms of viscosity.

### DISCLOSURE OF THE INVENTION

One subject of the present invention is thus a composition comprising:
a) at least one compound chosen from N,N-dicarboxymethylglutamic acid, salts thereof, solvates thereof, solvates of the salts thereof and mixtures thereof;
b) at least one colouring agent chosen from oxidation dyes, direct dyes and mixtures thereof,
c) at least one compound chosen from fatty amines, salts thereof and mixtures thereof.

According to a preferred embodiment, the composition according to the invention is a composition for dyeing keratin fibres, notably the hair.

The composition according to the invention may notably lead to chromatic, powerful, intense and sparingly selective colourings, i.e. colourings that are for example uniform along the length of the fibre, and in particular very good selectivity of the colourings. It also allows various shades to be achieved in a very wide range of colours. In addition, it enables good colour build-up and persistence.

Moreover, the composition according to the invention has good working qualities, notably a creamy texture, and easy and uniform spreading over the entire head of hair, and has good rheological properties, such as in terms of viscosity. The composition according to the invention has good stability over time, notably little or no change in its viscosity and/or its pH during storage.

The term “at least one” means one or more.

Unless otherwise indicated, the limits of a range of values are included in that range, notably in the expressions “between” and “ranging from . . . to . . . ”.

For the purposes of the present invention, the term “hair” means head hair. This term does not correspond to bodily hairs, the eyebrows or the eyelashes.

### N,N-Dicarboxymethylglutamic Acid

As indicated above, the composition according to the invention comprises at least one compound chosen from N,N-dicarboxymethylglutamic acid, salts thereof, solvates thereof, solvates of the salts thereof and mixtures thereof.

The salts are notably alkali metal, alkaline-earth metal, ammonium and substituted ammonium salts.

Among the salts of these compounds, the alkali metal salts and notably the sodium or potassium salts are preferred.

The composition according to the invention preferably comprises tetrasodium glutamate diacetate (GLDA). Use will be made, for example, of Dissolvine GL38 or 45S from AkzoNobel.

Advantageously, the total content of compounds chosen from N,N-dicarboxymethylglutamic acid, salts thereof, solvates thereof, solvates of the salts thereof and mixtures thereof ranges from 0.001% to 15% by weight, preferably from 0.005% to 10% by weight, more preferentially from 0.01% to 8% by weight, better still from 0.05% to 5% by weight, even better still from 0.075% to 2% by weight, or even from 0.1% to 1% by weight, relative to the total weight of the composition.

According to a preferred embodiment, the total content of tetrasodium glutamate diacetate (GLDA) ranges from 0.001% to 15% by weight, preferably from 0.005% to 10% by weight, more preferentially from 0.01% to 8% by weight, better still from 0.05% to 5% by weight, even better still from 0.075% to 2% by weight, or even from 0.1% to 1% by weight, relative to the total weight of the composition.

### Colouring Agent

As indicated above, the composition according to the invention comprises at least one colouring agent chosen from oxidation dyes, direct dyes and mixtures thereof.

### Oxidation Dye

The composition according to the invention may comprise one or more oxidation dyes.

The oxidation dyes are generally chosen from one or more oxidation bases optionally combined with one or more coupling agents (or couplers).

By way of example, the oxidation bases are chosen from para-phenylenediamines, bis(phenyl)alkylenediamines, para-aminophenols ortho-aminophenols and heterocyclic bases, and the corresponding addition salts.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-γ-hydroxypropyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl) pyrrolidine, and the corresponding addition salts with an acid.

Among the para-phenylenediamines mentioned above, para-phenylenediamine, para-toluenediamine, 2-β-hydroxyethyl-para-phenylenediamine, 2-methoxymethyl-para-phenylenediamine, 2-γ-hydroxypropyl-para-phenylenediamine, 2-isopropyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl) phenol and 4-amino-2-fluorophenol, and the corresponding addition salts with an acid.

Among the ortho-aminophenols that may be mentioned, for example, are 2-aminophenol, 2-amino-5-methylphenol, 2-amino-6-methylphenol and 5-acetamido-2-aminophenol, and the corresponding addition salts.

Among the heterocyclic bases that may be mentioned, for example, are pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl) pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl) methanol, 2-(3-aminopyrazolo[1,5-a]pyrid-5-yl) ethanol, 2-(3-aminopyrazolo[1,5-a]pyrid-7-yl) ethanol, (3-aminopyrazolo[1,5-a]pyrid-2-yl) methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl) pyrazolo[1,5-a]pyrid-3-ylamine, pyrazolo[1,5-a]pyridine-3,5-diamine, 5-(morpholin-4-yl) pyrazolo[1,5-a]pyrid-3-ylamine, 2-[(3-aminopyrazolo[1,5-a]pyrid-5-2-[(3-aminopyrazolo[1,5-a]pyrid-7-yl) (2-yl) (2-hydroxyethyl)amino]ethanol, hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine; and the corresponding addition salts.

More particularly, the oxidation bases that are useful in the present invention are chosen from 3-aminopyrazolo[1,5-a]pyridines preferably substituted on carbon atom 2 with:
a) a (di)(C₁-C₆) (alkyl)amino group, said alkyl group possibly being substituted with at least one hydroxyl, amino or imidazolium group;
b) an optionally cationic 5- to 7-membered heterocycloalkyl group comprising from 1 to 3 heteroatoms, optionally substituted with one or more (C₁-C₆)alkyl groups such as a di(C₁-C₄)alkylpiperazinium group; or
c) a (C₁-C₆)alkoxy group optionally substituted with one or more hydroxyl groups, such as a β-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(β-hydroxyethyl) pyrazole, 3,4-diaminopyrazole, 4,5-diamino-1-(4′-chlorobenzyl) pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl) pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts. Use may also be made of 4,5-diamino-1-(β-methoxyethyl) pyrazole.

A 4,5-diaminopyrazole will preferably be used and even more preferentially 4,5-diamino-1-(β-hydroxyethyl) pyrazole and/or a corresponding salt.

The pyrazole derivatives that may also be mentioned include diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino [1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Heterocyclic bases that will preferably be used are 4,5-diamino-1-(β-hydroxyethyl) pyrazole and/or 2,3-diamino-6,7-dihydro-1H,5H-pyrazolo[1,2-a]pyrazol-1-one and/or a corresponding salt.

Preferably, the oxidation base(s) are chosen from para-phenylenediamines, bis(phenyl)alkylenediamines, para-aminophenols, ortho-aminophenols, heterocyclic bases, and the corresponding addition salts, and mixtures thereof; more preferentially from 2-methoxymethyl-para-phenylenediamine, 2-β-hydroxyethyl-para-phenylenediamine, 2-γ-hydroxypropyl-para-phenylenediamine, and the addition salts thereof, the solvates thereof and/or the solvates of the salts thereof, and mixtures thereof.

The composition according to the invention may optionally comprise one or more coupling agents advantageously chosen from those conventionally used in the dyeing of keratin fibres.

Among these couplers, mention may be made in particular of meta-phenylenediamines, meta-aminophenols, meta-diphenols, naphthalene-based coupling agents and heterocyclic coupling agents, and also the corresponding addition salts.

Mention may be made, for example, of 6-hydroxybenzomorpholine, hydroxyethyl-3-4-methylenedioxyaniline, 2-amino-5-ethylphenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy) propane, 3-ureidoaniline, 3-ureido-1-dimethylaminobenzene, sesamol, α-naphthol, 2-methyl-1-naphthol, 6-hydroxyindole, 4-hydroxyindole, 4-hydroxy-N-methylindole, 2-amino-3-hydroxypyridine, 3,5-diamino-2,6-dimethoxypyridine, 1-N-(β-hydroxyethyl)amino-3,4-methylenedioxybenzene, 2,6-bis(β-hydroxyethylamino) toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid, and the corresponding mixtures.

Preferably, the coupler(s) are chosen from: 6-hydroxybenzomorpholine, the addition salts thereof, the solvates thereof and/or the solvates of the salts thereof, hydroxyethyl-3,4-methylenedioxyaniline, the addition salts thereof, the solvates thereof and/or the solvates of the salts thereof, 2-amino-5-ethylphenol, the addition salts thereof, the solvates thereof and/or the solvates of the salts thereof, and mixtures thereof.

In general, the addition salts of the oxidation bases and couplers that may be used in the context of the invention are chosen in particular from the addition salts with an acid such as the hydrochlorides, hydrobromides, sulfates, citrates, succinates, tartrates, lactates, tosylates, benzenesulfonates, phosphates and acetates.

Moreover, the solvates more particularly represent the hydrates of these oxidation bases or couplers and/or the combination of these oxidation bases or couplers with a linear or branched C1 to C4 alcohol such as methanol, ethanol, isopropanol or n-propanol. Preferably, the solvates are hydrates.

The oxidation base(s) each advantageously represent from 0.001% to 10% by weight relative to the total weight of the composition, and preferably from 0.005% to 5% by weight relative to the total weight of the composition and of the ready-to-use composition.

The coupler(s), if they are present, each advantageously represent from 0.001% to 10% by weight relative to the total weight of the composition, and preferably from 0.005% to 5% by weight relative to the total weight of the composition and of the ready-to-use composition.

### Direct Dye

The composition according to the invention may comprise at least one colouring agent chosen from direct dyes. Preferably, the colouring agent(s) are chosen from direct dyes.

The term “direct dye” means coloured species. These are dyes which will spread superficially over the fibre.

The direct dye(s) that may be used according to the invention are chosen from natural direct dyes, synthetic direct dyes, and mixtures thereof.

Preferably, the direct dye(s) that may be used according to the invention are chosen from ionic direct dyes and nonionic direct dyes, more particularly from cationic direct dyes, amphoteric direct dyes, anionic direct dyes, nonionic direct dyes and mixtures thereof.

The direct dyes are chosen, for example, from neutral, acidic or cationic nitrobenzene direct dyes, neutral, (nonionic), acidic (anionic) or cationic azo direct dyes, tetraazapentamethine dyes, neutral, acidic or cationic quinone and in particular anthraquinone dyes, azine direct dyes, triarylmethane direct dyes, azomethine direct dyes and natural direct dyes.

The cationic direct dye(s) contain at least one quaternized cationic chromophore or at least one chromophore bearing a quaternized or quaternizable cationic group.

According to a particular embodiment of the invention, the cationic direct dyes comprise at least one quaternized cationic chromophore.

As cationic direct dyes according to the invention, mention may be made of the following dyes: acridines; acridones; anthranthrones; anthrapyrimidines; anthraquinones; azines; (poly) azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines such as azacarbocyanines, diazacarbocyanines, diazahemicyanines, hemicyanines, or tetraazacarbocyanines; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; (poly) methines such as dimethines of stilbene or styryl type; naphthalimides; naphthanilides; naphtholactams; naphthoquinones; nitro, notably nitro (hetero) aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes, or xanthenes.

For the cationic azo dyes, mention may be made particularly of those resulting from the cationic dyes described in Kirk-Othmer's Encyclopedia of Chemical Technology, “Dyes, Azo”, J. Wiley & Sons, updated on 19 Apr. 2010.

Among the azo dyes that may be used according to the invention, mention may be made of the cationic azo dyes described in patent applications WO 95/15144, WO 95/01772 and EP714954.

According to a preferred embodiment of the invention, the direct dye(s) are chosen from cationic dyes known as “basic dyes”.

Among the azo dyes described in the Colour Index International 3rd edition, mention may be made notably of the following compounds:
Basic Red 22
Basic Red 76
Basic Yellow 57
Basic Brown 16
Basic Brown 17

Among the cationic quinone dyes, those mentioned in the abovementioned Colour Index International are suitable for use and, among these, mention may be made, inter alia, of the following dyes:
Basic Blue 22
Basic Blue 99.

Among the azine dyes that are suitable for use, mention may be made of those listed in the Colour Index International, for example the following dyes:
Basic Blue 17
Basic Red 2.

Among the cationic triarylmethane dyes that may be used according to the invention, mention may be made, in addition to those listed in the Colour Index, of the following dyes:
Basic Green 1
Basic Violet 3
Basic Violet 14
Basic Blue 7
Basic Blue 26.

Mention may also be made of the cationic dyes described in U.S. Pat. No. 5,888,252, EP 1 133 975, WO 03/029 359, EP 860 636, WO 95/01772, WO 95/15144 and EP 714 954. Mention may also be made of those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons.

Preferably, the cationic direct dyes are chosen from those resulting from dyes of azo and hydrazono type.

According to a particular embodiment, the direct dyes are cationic azo dyes, described in EP 850 636, FR 2 788 433, EP 920 856, WO 99/48465, FR 2 757 385, EP 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4 220 388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1 133 976, GB 738 585, DE 2 527 638, FR 2 275 462, GB 1974-27645, Acta Histochem. (1978), 61 (1), 48-52; Tsitologiya (1968), 10 (3), 403-5; Zh. Obshch. Khim. (1970), 40 (1), 195-202; Ann. Chim. (Rome) (1975), 65 (5-6), 305-14; Journal of the Chinese Chemical Society (Taipei) (1998), 45 (1), 209-211; Rev. Roum. Chim. (1988), 33 (4), 377-83; Text. Res. J. (1984), 54 (2), 105-7; Chim. Ind. (Milan) (1974), 56 (9), 600-3; Khim. Tekhnol. (1979), 22 (5), 548-53; Ger. Monatsh. Chem. (1975), 106 (3), 643-8; MRL Bull. Res. Dev. (1992), 6 (2), 21-7; Lihua Jianyan, Huaxue Fence (1993), 29 (4), 233-4; Dyes Pigm. (1992), 19 (1), 69-79; Dyes Pigm. (1989), 11 (3), 163-72.

Preferably, the cationic direct dye(s) comprise a quaternary ammonium group; more preferentially, the cationic charge is endocyclic.

These cationic radicals are, for example, a cationic radical:
bearing an exocyclic (di/tri) (C₁-C₈)alkylammonium charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic dyes of formulae (C-II) and (C-III), the azo dyes of formulae (C-IV) and (C-V) below, and also the optical and geometric isomers thereof and tautomers thereof, the organic or mineral acid or base salts thereof, and also the solvates thereof such as hydrates:

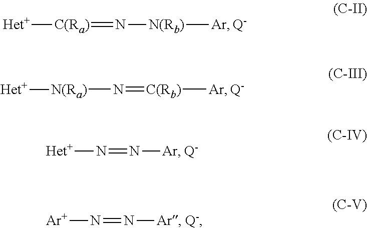

in which formulae (C-II) to (C—V):
   Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
   Ar⁺ represents an aryl radical, such as phenyl or naphthyl, bearing an exocyclic cationic charge, preferentially ammonium, particularly tri (C₁-C₈)alkylammonium, such as trimethylammonium;
   Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
   Ar″ represents an optionally substituted (hetero)aryl group, such as phenyl or pyrazolyl, which are optionally substituted, preferentially with one or more (C1-C8)alkyl, hydroxyl, (di)(C1-C8)(alkyl)amino, (C1-C8)alkoxy or phenyl groups;
   Rₐ and R_{b}, which may be identical or different, represent a hydrogen atom or a (C₁-C₈)alkyl group, which is optionally substituted, preferentially with a hydroxyl group;
   or else the substituent Rₐ with a substituent of Het⁺ and/or R_{b} with a substituent of Ar form, together with the atoms that bear them, a (hetero)cycloalkyl; in particular, Rₐ and R_{b} represent a hydrogen atom or a (C₁-C₄)alkyl group optionally substituted with a hydroxyl group;
   Q- represents an organic or mineral anionic counterion, such as a halide or an alkyl sulfate.

In particular, mention may be made of the azo and hydrazono direct dyes bearing endocyclic cationic charges, of formulae (C-II) to (C—V) as defined previously, more particularly the cationic direct dyes of formulae (C-II) to (C—V) bearing endocyclic cationic charges described in patent applications WO 95/15144, WO 95/01772 and EP-714954.

Preferentially, mention may be made of the following direct dyes:

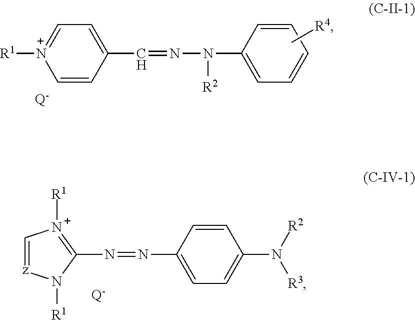

in which formulae (C-II-1) and (C-IV-1):
   R¹ represents a (C₁-C₄)alkyl group such as methyl;
   R² and R³, which may be identical or different, represent a hydrogen atom or a (C₁-C₄)alkyl group, such as methyl; and
   R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
   Z represents a CH group or a nitrogen atom, preferentially CH;
   Q⁻ is an anionic counterion as defined previously, in particular a halide, such as chloride, or an alkyl sulfate, such as methyl sulfate or mesyl.

In particular, the dyes of formulae (C-II-1) and (C-IV-1) are chosen from Basic Red 51, Basic Yellow 87 and Basic Orange 31 or derivatives thereof:

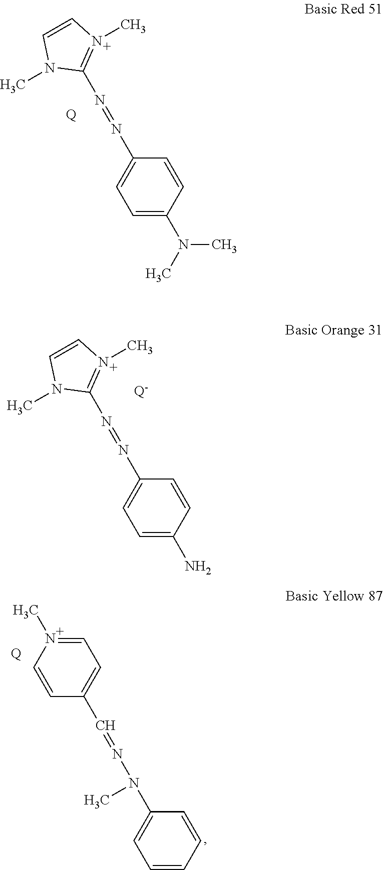

with Q′ being an anionic counterion as defined previously, in particular a halide, such as chloride, or an alkyl sulfate, such as methyl sulfate or mesyl.

According to a particular embodiment of the invention, the direct dyes are fluorescent, i.e. they contain at least one fluorescent chromophore as defined previously.

Fluorescent dyes that may be mentioned include the radicals resulting from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes and xanthenes.

Mention may also be made of the fluorescent dyes described in EP 1 133 975, WO 03/029 359, EP 860 636, WO 95/01772, WO 95/15144 and EP 714 954 and those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to one variant of the invention, the cationic dye(s) are fluorescent and comprise at least one quaternary ammonium radical such as those of formula (C-VI) below, and also the optical and geometric isomers thereof, the tautomers thereof, the organic or mineral acid or base salts thereof, and also the solvates thereof such as hydrates:

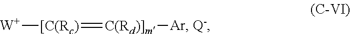

in which formula (C-VI):
   W⁺ represents a cationic heterocyclic or heteroaryl group, particularly comprising a quaternary ammonium optionally substituted with one or more (C₁-C₈)alkyl groups, optionally substituted notably with one or more hydroxyl groups;
   Ar representing an aryl group such as phenyl or naphthyl, optionally substituted preferentially with i) one or more halogen atoms such as chlorine or fluorine; ii) one or more groups (C₁-C₈)alkyl, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
   m represents an integer between 1 and 4 inclusive, and in particular m is 1 or 2; more preferentially 1;
   R_{c} and R_{d}, which may be identical or different, represent a hydrogen atom or an optionally substituted (C₁-C₈)alkyl group, preferentially a C₁-C₄ alkyl group, or alternatively R_{c} contiguous with W⁺ and/or R_{d} contiguous with Ar form, with the atoms that bear them, a (hetero)cycloalkyl; in particular, R_{c} is contiguous with W⁺ and they form a (hetero)cycloalkyl such as cyclohexyl;
   Q⁻ is an organic or mineral anionic counterion as defined previously.

Among the cationic direct dyes, mention may also be made of triarylmethane cationic dyes.

Preferably, the triarylmethane cationic direct dye(s) according to the invention are chosen from the cationic dyes of formulae (C-VII) and (C-VII′) below:

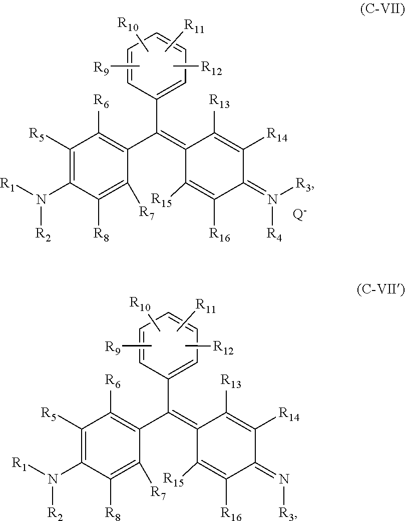

and also the organic or mineral acid or base addition salts thereof, the geometrical isomers, optical isomers and tautomers thereof, and the mesomeric forms thereof, and the solvates thereof such as hydrates:
in which formulae (C-VII) and (C-VII′):
   R₁, R₂, R₃ and R₄, which may be identical or different, represent a hydrogen atom or group from among: (C₁-C₆)alkyl which is optionally substituted, preferably with a hydroxyl group; aryl such as phenyl, aryl(C₁-C₄)alkyl such as benzyl, heteroaryl, heteroaryl(C₁-C₄)alkyl, or else two groups R₁, and R₂, and/or R₃ and R₄, borne by the same nitrogen atom, form, together with the nitrogen atom which bears them, an optionally substituted heterocycloalkyl group such as morpholino, piperazino or piperidino; preferably, R₁, R₂, R₃ and R₄, which may be identical or different, represent a hydrogen atom or a (C₁-C₄)alkyl group;
   R₅, R₆, R₇, R₈, R₉, R₁₀, R₁₁, R₁₂, R₁₃, R₁₄, R₁₅ and R₁₆, which may be identical or different, represent a hydrogen or halogen atom or a group chosen from i) hydroxyl, ii) thiol, iii) amino iv) (di)(C₁-C₄)(alkyl)amino, v) (di)arylamino such as (di)phenylamino, vi) nitro, vii) acylamino (—NR—C(O)R′) in which the radical R is a hydrogen atom, a C₁-C₄ alkyl radical optionally bearing at least one hydroxyl group and the radical R′ is a C₁-C₂ alkyl radical; viii) carbamoyl ((R)₂N—C(O)—) in which the radicals R, which may be identical or different, represent a hydrogen atom or a C₁-C₄ alkyl radical optionally bearing at least one hydroxyl group; ix) carboxylic acid or ester, (—O—C(O)R′) or
(—C(O)OR′), in which the radical R′ is a hydrogen atom or a C₁-C₄ alkyl optionally bearing at least one hydroxyl group and the radical R′ is a C₁-C₂ alkyl radical; x) alkyl optionally substituted notably with a hydroxyl group; xi) alkylsulfonylamino (R′SO₂—NR—) in which the radical R represents a hydrogen atom or a C₁-C₄ alkyl radical optionally bearing at least one hydroxyl group and the radical R′ represents a C₁-C₄ alkyl radical or a phenyl radical; xii) aminosulfonyl ((R)₂N—SO₂—) in which the radicals R, which may be identical or different, represent a hydrogen atom or a C₁-C₄ alkyl radical optionally bearing at least one hydroxyl group, xiii) (C₁-C₄)alkoxy, and xiv) (C₁-C₄)alkylthio;
   or else two radicals borne by two contiguous carbon atoms R₅ and R₆ and/or R₇ and R₈ and/or R₉ and R₁₀ and/or R₁₁ and R₁₂ and/or R₁₃ and R₁₄ and/or R₁₅ and R₁₆ form, together with the carbon atoms which bear them, an aryl or heteroaryl, preferably benzo, 6-membered fused ring, said ring possibly also being optionally substituted, preferably an unsubstituted benzo ring;
   Q⁻ represents an anionic counterion for achieving electrical neutrality, preferably chosen from halides such as chloride or bromide, and phosphate.

When the cationic dye comprises one or more anionic substituents such as COOR or SO₃R with R denoting a hydrogen or a cation, it is understood that there are then more cationic substituents than anionic substituents, such that the overall resulting charge of the triarylmethane structure is cationic.

According to a preferred embodiment, the triarylmethane dye(s) of the invention are chosen from those of formula (C-VII) or (C-VII′), in which, taken together or separately,
R₁, R₂, R₃ and R₄ represent a hydrogen atom or a (C₁-C₄)alkyl group such as methyl or ethyl;
R₅, R₆, R₇, R₈, R₉, R₁₀, R₁₁, R₁₂, R₁₃, R₁₄, R₁₅ and R₁₆ represent a hydrogen atom, a halogen atom, such as chlorine, or a (C₁-C₄)alkyl group such as methyl or ethyl, an amino group, a (di)(C₁-C₄)(alkyl)amino group and, preferably, at least one of the groups R₉, R₁₀, R₁₁ or R₁₂ represents a hydrogen atom, a halogen atom (Cl), or an amino group, or a (C₁-C₄)(alkyl)amino or (di)(C₁-C₄)(alkyl)amino group, preferably in the para position relative to the phenyl group.

Preferably, the direct dye(s) of triarylmethane structure are chosen from Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), and mixtures thereof.

The composition according to the invention may comprise one or more anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid acid dyes and natural acid dyes.

According to the invention, the anionic direct dye(s) may be chosen, alone or as a mixture, from the anionic direct dyes of formulae (A-II), (A-II′), (A-III), (A-III′), (A-IV), (A-IV′), (A-V), (A-V′), (A-VI), (A-VII), (A-VIII) and (A-IX) below:

### a) The Diaryl Anionic Azo Dyes of Formula (A-II) or (A-II′)

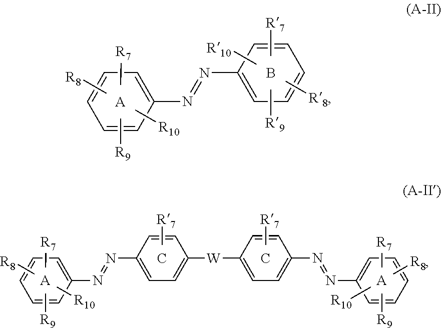

in which formulae (A-II) and (A-II′):
   R₇, R₈, R₉, R₁₀, R′₇, R′₈, R′₉ and R′₁₀, which may be identical or different, represent a hydrogen atom or a group chosen from:
   alkyl;
   alkoxy, alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   R″—S(O)₂—, with R″ representing a hydrogen atom or an alkyl group, an aryl, (di)(alkyl)amino or aryl(alkyl)amino group; preferentially a phenylamino or phenyl group;
   R′″—S(O)₂—X′— with R″′ representing an optionally substituted alkyl or aryl group, X′ as defined previously;
   (di)(alkyl)amino;
   aryl(alkyl)amino optionally substituted with one or more groups chosen from i) nitro; ii) nitroso; iii) (O)₂S(O⁻)—, M⁺ and iv) alkoxy with M⁺ as defined previously;
   optionally substituted heteroaryl; preferentially a benzothiazolyl group;
   cycloalkyl; notably cyclohexyl;
   Ar—N═N— with Ar representing an optionally substituted aryl group; preferentially a phenyl optionally substituted with one or more alkyl, (O)₂S(O⁻)—, M⁺ or phenylamino groups;
   or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁻; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(alkyl)amino; with M⁺, R^{o}, X, X′, X″ and Ar as defined previously;
   W represents a sigma bond a, an oxygen or sulfur atom, or a divalent radical i) —NR— with R as defined previously, or ii) methylene —C(Rₐ)(R_{b})— with Rₐ and R_{b}, which may be identical or different, representing a hydrogen atom or an aryl group, or alternatively Rₐ and R_{b} together form, with the carbon atom that bears them, a spiro cycloalkyl; preferentially, W represents a sulfur atom or Rₐ and R_{b} together form a cyclohexyl;
it being understood that formulae (A-II) and (A-II′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (A-II), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;
and, as examples of dyes of formula (A-II′), mention may be made of: Acid Red 111, Acid Red 134, Acid Yellow 38;

### b) The Pyrazolone Anionic Azo Dyes of Formulae (A-III) and (A-III′)

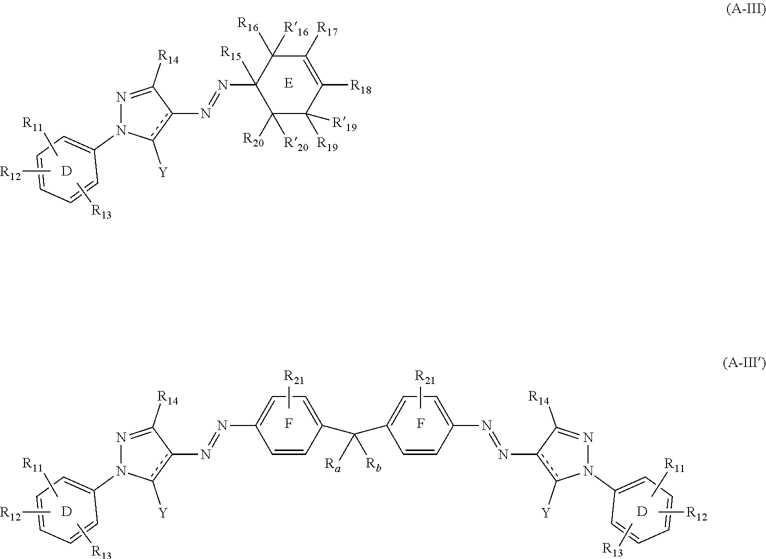

in which formulae (A-III) and (A-III′):
   R₁₁, R₁₂ and R₁₃, which may be identical or different, represent a hydrogen or halogen atom, an alkyl group or -(O)₂S(O⁻), M⁺ with M⁺ as defined previously;
   R₁₄ represents a hydrogen atom, an alkyl group or a group —C(O)O—, M⁺ with M⁺ as defined previously;
   R₁₅ represents a hydrogen atom;
   R₁₆ represents an oxo group, in which case R′₁₆ is absent, or alternatively R₁₅ with R₁₆ together form a double bond;
   R₁₇ and R₁₈, which may be identical or different, represent a hydrogen atom, or a group chosen from:
   (O)₂S(O⁻)—, M⁺ with M⁺ as defined previously;
   Ar—O—S(O)₂— with Ar representing an optionally substituted aryl group; preferentially a phenyl optionally substituted with one or more alkyl groups;
   R₁₉ and R₂₀ together form either a double bond, or a benzo group D′, which is optionally substituted;
   R′₁₆, R′₁₉ and R′₂₀, which may be identical or different, represent a hydrogen atom or an alkyl or hydroxyl group;
   R₂₁ represents a hydrogen atom or an alkyl or alkoxy group;
   Rₐ and R_{b}, which may be identical or different, are as defined previously; preferentially, Rₐ represents a hydrogen atom and R_{b} represents an aryl group;
   Y represents either a hydroxyl group or an oxo group: [symbol]
      represents a single bond when Y is an oxo group; and represents a double bond when Y represents a hydroxyl group;
it being understood that formulae (A-III) and (A-III′) comprise at least one sulfonate radical (O)₂S(O⁻)—, M⁺ or one carboxylate radical —C(O)O⁻, M⁺ on one of the rings D or E; preferentially sodium sulfonate.

As examples of dyes of formula (A-III), mention may be made of: Acid Red 195, Acid Yellow 23, Acid Yellow 27, Acid Yellow 76, and as examples of dyes of formula (A-III′), mention may be made of: Acid Yellow 17;

### c) The Anthraquinone Dyes of Formulae (A-IV) and (A-IV′)

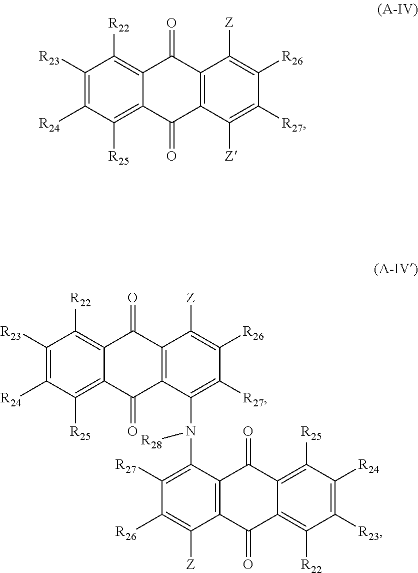

in which formulae (A-IV) and (A-IV′):
   R₂₂, R₂₃, R₂₄, R₂₅, R₂₆ and R₂₇, which may be identical or different, represent a hydrogen or halogen atom, or a group chosen from:
   alkyl;
   hydroxyl, mercapto;
   alkoxy, alkylthio;
   optionally substituted aryloxy or arylthio, preferentially substituted with one or more groups chosen from alkyl and (O)₂S(O⁻)—, M⁺ with M⁺ as defined previously;
   aryl(alkyl)amino optionally substituted with one or more groups chosen from alkyl and (O)₂S(O⁻)—, M⁺ with M⁺ as defined previously;
   (di)(alkyl)amino;
   (di)(hydroxyalkyl)amino;
   (O)₂S(O⁻)—, M⁺ with M⁺ as defined previously;
   Z′ represents a hydrogen atom or a group NR₂₈R₂₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an alkyl group;
   cycloalkyl, notably cyclohexyl;
   Z represents a group chosen from hydroxyl and NR′₂₈R′₂₉ with R′₂₈ and R′₂₉, which may be identical or different, representing the same atoms or groups as R₂₈ and R₂₉ as defined previously;
it being understood that formulae (A-IV) and (A-IV′) comprise at least one sulfonate radical (O)₂S(O⁻)—, M⁺ or one carboxylate radical —C(O)O—, M⁺; preferentially sodium sulfonate.

As examples of dyes of formula (A-IV), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2;
and, as examples of dyes of formula (A-IV′), mention may be made of: Acid Black 48;

### d) The Nitro Dyes of Formulae (A-V) and (A-V′)

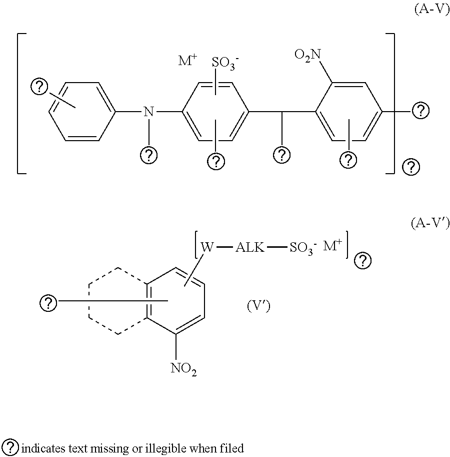

in which formulae (A-V) and (A-V′):
   R₃₀, R₃₁ and R₃₂, which may be identical or different, represent a hydrogen or halogen atom, or a group chosen from:
   alkyl;
   alkoxy optionally substituted with one or more hydroxyl groups, alkylthio optionally substituted with one or more hydroxyl groups;
   hydroxyl, mercapto;
   nitro, nitroso;
   polyhaloalkyl;
   R^{o}—C(X)—X′—, R^{o}—X′—C(X)—, R^{o}—X′—C(X)—X″— with R^{o}, X, X′ and X″ as defined previously;
   (O)₂S(O⁻)—, M⁺ with M⁺ as defined previously;
   (O)CO⁻—, M⁺ with M⁺ as defined previously;
   (di)(alkyl)amino;
   (di)(hydroxyalkyl)amino;
   heterocycloalkyl such as piperidino, piperazino or morpholino;
in particular, R₃₀, R₃₁ and R₃₂ represent a hydrogen atom;
   R_{c} and R_{d}, which may be identical or different, represent a hydrogen atom or an alkyl group;
   W is as defined previously; W particularly represents an —NH— group;
   ALK represents a linear or branched divalent C₁-C₆ alkylene group; in particular, ALK represents a —CH₂—CH₂— group;
   n is 1 or 2;
   p represents an integer inclusively between 1 and 5;
   q represents an integer inclusively between 1 and 4;
   u is 0 or 1;
   when n is 1, J represents a nitro or nitroso group; particularly nitro;
   when n is 2, J represents an oxygen or sulfur atom, or a divalent radical —S(O)ₘ— with m representing an integer 1 or 2; preferentially, J represents an —SO₂-radical;
   M′ represents a hydrogen atom or a cationic counterion;

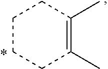

which may be present or absent, represents a benzo group optionally substituted with one or more groups R₃₀ as defined previously;
it being understood that formulae (A-V) and (A-V′) comprise at least one sulfonate radical (O)₂S(O⁻)—, M⁺ or one carboxylate radical —C(O)O⁻, M⁺; preferentially sodium sulfonate.

As examples of dyes of formula (A-V), mention may be made of: Acid Brown 13 and Acid Orange 3; as examples of dyes of formula (A-V′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-p-hydroxyethylamino-3-nitrobenzenesulfonic acid; EXT D&C Yellow 7;

### e) The Triarylmethane Dyes of Formula (A-VI)

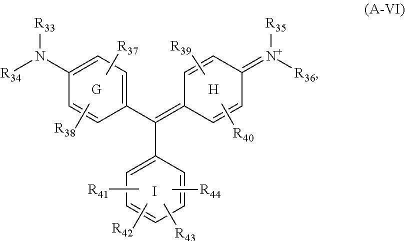

in which formula (A-VI):
   R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
   R₃₇, R₃₈, R₃₉, R₄₀, R₄₁, R₄₂, R₄₃ and R₄₄, which may be identical or different, represent a hydrogen atom or a group chosen from:
   alkyl;
   alkoxy, alkylthio;
   (di)(alkyl)amino;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M; iv) hydroxyl; v) mercapto; vi) (di)(alkyl)amino; vii) R^{o}—C(X)—X′—; viii) R^{o}—X′—C(X)— and ix) R^{o}—X′—C(X)—X″—; with M⁺, R^{o}, X, X′ and X″ as defined previously;
in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
it being understood that at least one of the rings G, H, I or I′ comprises at least one sulfonate radical (O)₂S(O—)- or one carboxylate radical —C(O)O⁻; preferentially sulfonate.

As examples of dyes of formula (A-VI), mention may be made of: Acid Blue 1; Acid Blue 3; Acid Blue 7, Acid Blue 9; Acid Violet 49; Acid Green 3; Acid Green 5 and Acid Green 50;

### f) The Xanthene-Based Dyes of Formula (A-VII)

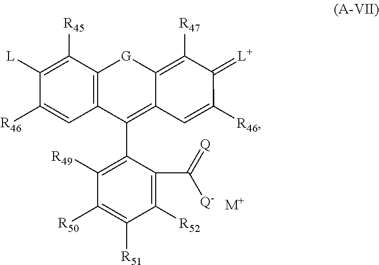

in which formula (A VII)
   R₄₅, R₄₆, R₄₇ and R₄₈, which may be identical or different, represent a hydrogen or halogen atom;
   R₄₉, R₅₀, R₅₁ and R₅₂, which may be identical or different, represent a hydrogen or halogen atom, or a group chosen from:
   alkyl;
   alkoxy, alkylthio;
   hydroxyl, mercapto;
   nitro, nitroso;
   (O)₂S(O⁻)—, M⁺ with M⁺ representing a hydrogen atom or a cationic counterion;
   (O)CO⁻—, M⁺ with M⁺ as defined previously;
particularly, R₅₃, R₅₄, R₅₅ and R₄₈ represent a hydrogen or halogen atom;
   G represents an oxygen or sulfur atom or a group NRₑ with Rₑ as defined previously; particularly, G represents an oxygen atom;
   L represents an alkoxide O⁻, M⁺; a thioalkoxide S⁻, M⁺ or a group NR_{f}, with R_{f} representing a hydrogen atom or an alkyl group and M⁺ as defined previously; M⁺ is particularly sodium or potassium;
   L′ represents an oxygen or sulfur atom or an ammonium group: N⁺ R_{f}R_{g}, with R_{f} and R_{g}, which may be identical or different, representing a hydrogen atom or an optionally substituted alkyl or aryl group; L′ particularly represents an oxygen atom or a phenylamino group optionally substituted with one or more alkyl or (O)ₘS(O—)-, M⁺ groups with m and M⁺ as defined previously;
   Q and Q′, which may be identical or different, represent an oxygen or sulfur atom; particularly, Q and Q′ represent an oxygen atom;
   M⁺ is as defined previously.

As examples of dyes of formula (A-VII), mention may be made of: Acid Yellow 73; Acid Red 51; Acid Red 52; Acid Red 87; Acid Red 92; Acid Red 95; Acid Violet 9;

### g) The Indole-Based Dyes of Formula (A-VIII)

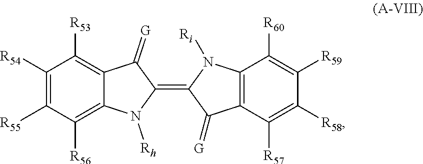

in which formula (A-VIII):
   R₅₃, R₅₄, R₅₅, R₅₆, R₅₇, R₅₈, R₅₉ and R₆₀, which may be identical or different, represent a hydrogen atom or a group chosen from:
   alkyl;
   alkoxy, alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   G represents an oxygen or sulfur atom or a group NRₑ with Rₑ as defined previously; particularly, G represents an oxygen atom;
   Rᵢ and Rₕ, which may be identical or different, represent a hydrogen atom or an alkyl group;
it being understood that formula (A-VIII) comprises at least one sulfonate radical (O)₂S(O⁻)—, M⁺ or one carboxylate radical —C(O)O—, M⁺; preferentially sodium sulfonate.

As examples of dyes of formula (A-VIII), mention may be made of: Acid Blue 74.

### h) The Quinoline-Based Dyes of Formula (A-IX)

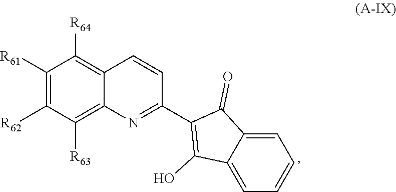

in which formula (A-IX):
   R₆₁ represents a hydrogen or halogen atom or an alkyl group;
   R₆₂, R₆₃ and R₆₄, which may be identical or different, represent a hydrogen atom or a group (O)₂S(O⁻)—, M⁺ with M⁺ representing a hydrogen atom or a cationic counterion;
   or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
it being understood that formula (A-X) comprises at least one sulfonate radical (O)₂S(O⁻)—, M⁺, preferentially sodium sulfonate.

As examples of dyes of formula (A-IX), mention may be made of: Acid Yellow 2, Acid Yellow 3 and Acid Yellow 5.

More particularly, the dye composition comprises one or more anionic direct dyes chosen, alone or as a mixture, from the following anionic direct dyes:

TABLE 1

| | |
|---|---|
| (C.I. 45380) | Acid Red 87 (A-VII) |
| (C.l. 10316) | Sodium salt of 2,4-dinitro-1-naphthol-7-sulfonic acid (A-V′) |
| (C.l. 10383) | Acid Orange 3 (A-V) |
| (C.l. 13015) | Acid Yellow 9/Food Yellow 2 (A-II) |
| (C.l. 14780) | /Direct Red 45/Food Red 13 (A-II) |
| (C.l. 13711) | Acid Black 52 (A-II) |
| (C.l. 13065) | Acid Yellow 36 (A-II) |
| (C.l. 14700) | Sodium salt of 1-hydroxy-2-(2′,4′-xylyl-5-sulfonatoazo) |
| | naphthalene-4-sulfonic acid/Food Red 1 (A-II) |
| (C.l. 14720) | Acid Red 14/Food Red 3/Mordant Blue 79 (A-II) |
| (C.l. 14805) | Sodium salt of 4-hydroxy-3-[(2-methoxy-5-nitrophenyl) |
| | diaza]-6-(phenylamino)naphthalene-2-sulfonic acid/Acid |
| | Brown 4 (A-II) |
| (C.l. 15510) | Acid Orange 7/Pigment Orange 17/Solvent Orange |
| | 49 (A-II) |
| (C.l. 15985) | Food Yellow 3/Pigment Yellow 104 (A-II) |
| (C.l. 16185) | Acid Red 27/Food Red 9 (A-II) |
| (C.l. 16230) | Acid Orange 10/Food Orange 4 (A-II) |
| (C.l. 16250) | Acid Red 44 (A-II) |
| (C.l. 17200) | Acid Red 33/Food Red 12 (A-II) |
| (C.l. 15685) | Acid Red 184 (A-II) |
| (C.l. 19125) | Acid Violet 3 (A-II) |
| (C.I. 18055) | Sodium salt of 1-hydroxy-2-(4′-acetamidophenylazo)-8- |
| | acetamidonaphthalene-3,6-disulfonic acid/Acid Violet |
| | 7/Food Red 11 (A-II) |
| (C.l. 18130) | Acid Red 135 (A-II) |
| (C.l. 19130) | Acid Yellow 27 (A-III) |
| (C.l. 19140) | Acid Yellow 23/Food Yellow 4 (A-III) |
| (C.l. 20170) | 4′-(Sulfonato-2″,4″-dimethyl)bis(2,6-phenylazo)-1,3- |
| | dihydroxybenzene/Acid Orange 24 (A-II) |
| (C.l. 20470) | Sodium salt of 1-amino-2-(4′-nitrophenylazo)-7- |
| | phenylazo-8-hydroxynaphthalene-3,6-disulfonic acid/Acid |
| | Black 1 (A-II) |
| (C.l. 23266) | (4-((4-Methylphenyl)sulfonyloxy)phenylazo)-2,2′-dimethyl- |
| | 4-((2-hydroxy-5,8-disulfonato)naphthylazo)biphenyl/Acid |
| | Red 111 (A-II′) |
| (C.l. 27755) | Food Black 2 (A-II) |
| (C.l. 25440) | 1-(4'-Sulfonatophenylazo)-4-((2″-hydroxy-3″-acetylamino- |
| | 6″,8″-disulfonato)naphthylazo)-6-sulfonatonaphthalene |
| | (tetrasodium salt)/Food Black 1 (A-II) |
| (C.I. 42090) | Acid Blue 9 (A-VI) |
| (C.I. 60730) | Acid Violet 43 (A-IV) |
| (C.I. 61570) | Acid Green 25 (A-IV) |
| (C.I. 62045) | Sodium salt of 1-amino-4-cyclohexylamino-9,10- |
| | anthraquinone-2-sulfonic acid/Acid Blue 62 (A-IV) |
| (C.I. 62105) | Acid Blue 78 (A-IV) |
| (C.l. 14710) | Sodium salt of 4-hydroxy-3-((2-methoxyphenyl)azo)-1- |
| | naphthalenesulfonic acid/Acid Red 4 (A-II) |
| | 2-Piperidino-5-nitrobenzenesulfonic acid (V′) |
| | 2-(4′-N,N-(2″-Hydroxyethyl)amino-2′- |
| | nitro)anilineethanesulfonic acid (A-V′) |
| | 4-β-Hydroxyethylamino-3-nitrobenzene |
| | sulfonic acid (A-V′) |
| (C.I. 42640) | Acid Violet 49 (A-VI) |
| (C.I. 42080) | Acid Blue 7 (A-VI) |
| (C.I. 58005) | Sodium salt of 1,2-dihydroxy-3-sulfoanthraquinone/ |
| | Mordant Red 3 (A-IV) |
| (C.I. 62055) | Sodium salt of 1-amino-9,10-dihydro-9,10-dioxo-4- |
| | (phenylamino)-2-anthracenesulfonic acid/Acid Blue |
| | 25 (A-IV) |
| (C.I. 14710) | Sodium salt of 4-hydroxy-3-((2-methoxyphenyl)azo)-1- |
| | naphthalenesulfonic acid/Acid Red 4 (A-II) |

Most of these dyes are described in particular in the Colour Index published by The Society of Dyers and Colourists, P.O. Box 244, Perkin House, 82 Grattan Road, Bradford, Yorkshire, BD1 2JB, England.

The anionic dyes which are most particularly preferred are the dyes designated in the Colour Index under the code C.I. 58005 (monosodium salt of 1,2-dihydroxy-9,10-anthraquinone-3-sulfonic acid), C.I. 60730 (monosodium salt of 2-[(9,10-dihydro-4-hydroxy-9,10-dioxo-1-anthracenyl)amino]-5-methylbenzenesulfonic acid), C.I. 15510 (monosodium salt of 4-[(2-hydroxy-1-naphthalenyl)azo]benzenesulfonic acid), C.I. 15985 (disodium salt of 6-hydroxy-5-[(4-sulfophenyl)azo]-2-naphthalenesulfonic acid), C.I. 17200 (disodium salt of 5-amino-4-hydroxy-3-(phenylazo)-2,7-naphthalenedisulfonic acid), C.I. 20470 (disodium salt of 1-amino-2-(4′-nitrophenylazo)-7-phenylazo-8-hydroxy-3,6-naphthalenedisulfonic acid), C.I. 42090 (disodium salt of N-ethyl-N-[4-[[4-[ethyl[(3-sulfophenyl)methyl]amino]phenyl](2-sulfophenyl)methylene]-2,5-cyclohexadien-1-ylidene]-3-sulfobenzenemethanaminium hydroxide, internal salt), C.I. 61570 (disodium salt of 2,2′-[(9,10-dihydro-9,10-dioxo-1,4-anthracenediyl)diimino]bis[5-methyl]benzenesulfonic acid).

Use may also be made of compounds corresponding to the mesomeric or tautomeric forms of structures (A-II) to (A-IX).

Among the natural direct dyes that may be used according to the invention, mention may be made of hennotannic acid, juglone, alizarin, purpurin, carminic acid, kermesic acid, purpurogallin, protocatechaldehyde, indigo, isatin, curcumin, spinulosin, apigenidin and orcein. Extracts or decoctions containing these natural dyes and in particular henna-based poultices or extracts may also be used.

Preferably, the colouring agent is chosen from direct dyes; more preferentially, said direct dye(s) are chosen from ionic direct dyes and nonionic direct dyes, more particularly from cationic direct dyes, amphoteric direct dyes, anionic direct dyes, nonionic direct dyes and mixtures thereof.

Advantageously, the colouring agent is chosen from direct dyes, more preferentially chosen from azo direct dyes, hydrazono direct dyes, nitroaryl direct dyes, triarylmethane direct dyes, quinone direct dyes and in particular anthraquinone direct dyes, and mixtures thereof.

Advantageously, the total content of the colouring agent(s), preferably of the direct dye(s), ranges from 0.001% to 20% by weight, preferably from 0.005% to 15% by weight, more preferentially from 0.01% to 10% by weight, better still from 0.05% to 5% and even better still from 0.1% to 3% by weight, relative to the total weight of the composition.

### Fatty Amine

As indicated above, the composition according to the invention comprises at least one compound chosen from fatty amines, salts thereof and mixtures thereof.

The term “fatty amine” means a compound comprising at least one optionally (poly)oxyalkylenated primary, secondary or tertiary amine function, or salts thereof and comprising at least one C₆-C₃₀ and preferably C₈-C₃₀ hydrocarbon-based chain.

Preferably, the fatty amines that are useful according to the invention are not (poly)oxyalkylenated.

Among the fatty amines, mention may be made of stearylamine, stearyldimethylamine and distearylamine.

Fatty amines that may also be mentioned include amidoamines. The amidoamines according to the invention may be chosen from fatty amidoamines, it being possible for the fatty chain to be borne by the amine group or by the amido group.

The term “amidoamine” means a compound comprising at least one amide function and at least one primary, secondary or tertiary amine function.

The term “fatty amidoamine” means an amidoamine comprising at least one C₆-C₃₀ hydrocarbon-based chain. Preferably, the fatty amidoamines that are useful according to the invention are not quaternized.

Preferably, the fatty amidoamines that are useful according to the invention are not (poly)oxyalkylenated.

Preferably, the fatty amidoamines are chosen from (C₁₀-C₃₀)alkylamido(C₁-C₈)alkyl (di)(C₁-C₆)alkyl amines and salts thereof, better still from (C₁₄-C₂₆)alkylamido(C₁-C₆)alkyl (di)(C₁-C₄)alkyl amines and salts thereof, preferentially from (C₁₆-C₂₄)alkylamido(C₂-C₄)alkyl (di)(C₁-C₂)alkyl amines and salts thereof; even more preferentially from (C₁₈-C₂₂)alkylamido(C₂-C₄)alkyl (di)(C₁-C₂)alkyl amines and salts thereof.

Among the fatty amidoamines that are useful according to the invention, mention may be made of the amidoamines of formula (A) below: RCONHR″N(R′)₂ (A), in which:
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C₈-C₂₉ and preferably C₇-C₂₃ alkyl radical, or a linear or branched C₈-C₂₉ and preferably C₇-C₂₃ alkenyl radical;
R″ represents a divalent hydrocarbon-based radical containing less than 6 carbon atoms, preferably from 2 to 4 carbon atoms and better still 3 carbon atoms; and
R′, which may be identical or different, represent a substituted or unsubstituted, saturated or unsaturated, linear or branched, monovalent hydrocarbon-based radical containing less than 6 carbon atoms, preferably from 1 to 4 carbon atoms, preferably a methyl radical.

As fatty amidoamines that may be used, mention may be made, alone or as a mixture, of oleamidopropyldimethylamine, stearamidopropyldimethylamine, notably the product sold by the company Inolex Chemical Company under the name Lexamine S13, isostearamidopropyldimethylamine, stearamidoethyldimethylamine, lauramidopropyldimethylamine, myristamidopropyldimethylamine, behenamidopropyldimethylamine, dilinoleamidopropyldimethylamine, palmitamidopropyldimethylamine, ricinoleamindopropyldimethylamine, soyamidopropyldimethylamine, avocadoamidopropyldimethylamine, cocamidopropyldimethylamine, minkamidopropyldimethylamine, oatamidopropyldimethylamine, sesamidopropyldimethylamine, tallamidopropyldimethylamine, olivamidopropyldimethylamine, palmitamidopropyldimethylamine, stearamidoethyldiethylamine, brassicamidopropyldimethylamine and salts thereof.

Preferably, the fatty amidoamines are chosen, alone or as mixtures, including the salts thereof, from:
brassicamidopropyldimethylamine having the formula R—C(O)—N(H)—(CH₂)₃—N(CH₃)₂;
in which R—C(O) is a fatty acid derived from Brassica campestris seed oil (rapeseed oil), with a majority of behenyl group (C₂₂);
stearamidopropyldimethylamine having the formula CH₃—(CH₂)₁₆—C(O)—N(H)—(CH₂)₃—N(CH₃)₂;
behenamidopropyldimethylamine having the formula CH₃—(CH₂)₂₀—C(O)—N(H)—(CH₂)₃—N(CH₃)₂;
oleamidopropyldimethylamine.

Advantageously, the fatty amines are chosen from fatty amidoamines, salts thereof and mixtures thereof, preferably from the amidoamines of formula (A).

Preferably, the amidoamines of formula (A) are chosen from brassicamidopropyldimethylamine, stearamidopropyldimethylamine, behenamidopropyldimethylamine, oleamidopropyldimethylamine, and mixtures thereof.

According to a preferred embodiment, the compound(s) chosen from fatty amines, salts thereof and mixtures thereof are chosen from fatty amidoamines, salts thereof and mixtures thereof, preferably from (C₁₀-C₃₀)alkylamido(C₁-C₈)alkyl (di)(C₁-C₆)alkyl amines and salts thereof, more preferentially from (C₁₄-C₂₆)alkylamido(C₁-C₆)alkyl (di)(C₁-C₄)alkyl amines and salts thereof, even more preferentially from (C₁₆-C₂₄)alkylamido(C₂-C₄)alkyl (di)(C₁-C₂)alkyl amines and salts thereof; better still from (C₁₈-C₂₂)alkylamido(C₂-C₄)alkyl (di)(C₁-C₂)alkyl amines and salts thereof.

According to a particularly preferred embodiment, the compound(s) chosen from fatty amines, salts thereof and mixtures thereof are chosen from brassicamidopropyldimethylamine, stearamidopropyldimethylamine, behenamidopropyldimethylamine, oleamidopropyldimethylamine and mixtures thereof.

Advantageously, the total content of the compound(s) chosen from fatty amines, salts thereof and mixtures thereof ranges from 0.01% to 6% by weight, preferably from 0.1% to 4.5% by weight, more preferentially from 1.5% to 3% by weight, relative to the total weight of the composition.

Advantageously, the total content of the compound(s) chosen from (C₁₀-C₃₀)alkylamido(C₁-C₈)alkyl (di)(C₁-C₆)alkylamines and salts thereof ranges from 0.01% to 6% by weight, preferably from 0.1% to 4.5% by weight, more preferentially from 1.5% to 3% by weight, relative to the total weight of the composition.

Advantageously, the total content of the compound(s) chosen from the amidoamines of formula (A) ranges from 0.01% to 6% by weight, preferably from 0.1% to 4.5% by weight, more preferentially from 1.5% to 3% by weight, relative to the total weight of the composition.

### Non-Silicone Fatty Substances

The composition according to the invention may also comprise at least one non-silicone fatty substance different from the fatty amines described above.

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). The fatty substances have in their structure at least one hydrocarbon-based chain including at least six carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The non-silicone fatty substances that may be used in the present invention, different from fatty amines, are neither (poly)oxyalkylenated nor (poly)glycerolated.

The term “non-silicone fatty substance” refers to a fatty substance not containing any Si—O bonds and the term “silicone fatty substance” refers to a fatty substance containing at least one Si—O bond.

Preferably, the non-silicone fatty substance(s) according to the invention, different from fatty amines, are different from fatty acids.

The non-silicone fatty substances that are useful according to the invention may be liquid non-silicone fatty substances (or oils) and/or solid non-silicone fatty substances and/or pasty non-silicone fatty substances. The term “liquid fatty substance” means a fatty substance with a melting point of less than or equal to 25° C. at atmospheric pressure (1.013×10⁵ Pa) and the term “solid fatty substance” means a fatty substance with a melting point of greater than 25° C. at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, all the melting points are determined at atmospheric pressure (1.013×10⁵ Pa).

More particularly, the liquid non-silicone fatty substance(s) may be chosen from C₆ to C₁₆ liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid fatty acid and/or fatty alcohol esters other than triglycerides, and mixtures thereof.

It is recalled that the fatty alcohols, esters and acids more particularly contain at least one saturated or unsaturated, linear or branched hydrocarbon-based group comprising from 6 to 40 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

As regards the C₆-C₁₆ liquid hydrocarbons, the latter may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly (or mineral oil), polydecenes, hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PCl and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, including from 6 to 40 carbon atoms and preferably from 8 to 30 carbon atoms. These fatty alcohols are neither oxyalkylenated nor glycerolated. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof. Preferably, oleyl alcohol will be used.

As regards the liquid fatty acid and/or fatty alcohol esters, other than the triglycerides mentioned previously, mention may be made notably of esters of saturated or unsaturated, linear C1 to C26 or branched C3 to C26 aliphatic mono- or polyacids and of saturated or unsaturated, linear C1 to C26 or branched C3 to C26 aliphatic mono- or polyalcohols, the total carbon number of the esters being greater than or equal to 6 and more advantageously greater than or equal to 10.

According to a preferred embodiment, the liquid fatty acid and/or fatty alcohol esters other than triglycerides are chosen from liquid fatty acid and fatty alcohol esters, preferably chosen from linear monoalcohol esters, more preferentially chosen from cocoyl caprylate, cocoyl caprate and mixtures thereof.

Preferably, for the esters of monoalcohols, at least one from among the alcohol and the acid is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, 2-ethylhexyl palmitate, 2-octyldecyl palmitate; alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Esters of C4 to C22 dicarboxylic or tricarboxylic acids and of C1 to C22 alcohols and esters of monocarboxylic, dicarboxylic or tricarboxylic acids and of C2 to C26 dihydroxy, trihydroxy, tetrahydroxy or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C6 to C30 and preferably C12 to C22 fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides other than the anionic polysaccharides described previously.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described above and of linear or branched, saturated or unsaturated C6 to C30 and preferably C12 to C22 fatty acids. If they are unsaturated, these compounds may comprise one to three conjugated or unconjugated carbon-carbon double bonds.

The esters may also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

Preferably, the composition according to the invention comprises a liquid monoacid and monoalcohol ester.

According to one embodiment, the non-silicone fatty substances that are useful according to the invention are chosen from liquid fatty substances, preferably from C₆ to C₁₆ liquid hydrocarbons, plant oils, liquid hydrocarbons containing more than 16 carbon atoms, liquid fatty alcohols, liquid fatty acid and/or fatty alcohol esters other than triglycerides and mixtures thereof.

The solid fatty substances preferably have a viscosity of greater than 2 Pa·s, measured at 25° C. and at a shear rate of 1 s⁻¹.

The solid fatty substance(s) are preferably chosen from solid fatty alcohols, solid fatty acid and/or fatty alcohol esters, waxes, ceramides and mixtures thereof.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 10 to 30, or even from 12 to 24 atoms and even better still from 14 to 22 carbon atoms.

The solid fatty alcohols that may be used are preferably chosen from saturated or unsaturated, linear or branched, preferably linear and saturated, (mono)alcohols including from 8 to 40 carbon atoms, better still from 10 to 30, or even from 12 to 24 and even better still from 14 to 22 carbon atoms.

The solid fatty alcohols that may be used may be chosen, alone or as a mixture, from: myristyl alcohol (or 1-tetradecanol); cetyl alcohol (or 1-hexadecanol); stearyl alcohol (or 1-octadecanol); arachidyl alcohol (or 1-eicosanol); behenyl alcohol (or 1-docosanol); lignoceryl alcohol (or 1-tetracosanol); ceryl alcohol (or 1-hexacosanol); montanyl alcohol (or 1-octacosanol); myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl alcohol or cetearyl alcohol.

Particularly preferably, the solid fatty alcohol is chosen from cetyl alcohol, stearyl alcohol and mixtures thereof such as cetylstearyl alcohol or cetearyl alcohol.

The solid esters of a fatty acid and/or of a fatty alcohol that may be used are preferably chosen from esters derived from a C9-C26 carboxylic fatty acid and/or from a C9-C26 fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C4-C22 dicarboxylic or tricarboxylic acids and of C1-C22 alcohols and esters of monocarboxylic, dicarboxylic or tricarboxylic acids and of C2-C26 dihydroxy, trihydroxy, tetrahydroxy or pentahydroxy alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl, cetyl or stearyl palmitate; C₉-C₂₆ alkyl myristates, such as cetyl myristate, stearyl myristate and myristyl myristate; and C₉-C₂₆ alkyl stearates, notably myristyl, cetyl and stearyl stearate; and mixtures thereof.

For the purposes of the present invention, a wax is a lipophilic compound, which is solid at 25° C. and atmospheric pressure, with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may be made notably of hydrocarbon-based waxes, for instance beeswax, notably of organic origin, lanolin wax and Chinese insect waxes; rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of C20 to C60 microcrystalline waxes, such as Microwax HW.

Mention may also be made of the MW 500 polyethylene wax sold under the reference Permalen 50-L Polyethylene.

Mention may also be made of the waxes obtained by catalytic hydrogenation of animal or plant oils containing linear or branched C8-C32 fatty chains. Among these waxes, mention may notably be made of isomerized jojoba oil such as trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, notably the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may also be used is a C20 to C40 alkyl (hydroxystearyloxy)stearate (the alkyl group comprising from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may notably be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin wax, petroleum jelly wax, lignite wax or ozokerite; plant waxes, for instance cocoa butter or cork fibre or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

The ceramides or ceramide analogues, such as glycoceramides, that may be used in the compositions according to the invention, are known; mention may in particular be made of ceramides of classes I, II, III and V according to the Dawning classification.

The ceramides or analogues thereof that may be used preferably correspond to the following formula: R³CH(OH)CH(CH₂OR²)(NHCOR¹), in which:
R¹ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R² denotes a hydrogen atom, a (glycosyl)ₙ group, a (galactosyl)ₘ group or a sulfogalactosyl group, in which n is an integer ranging from 1 to 4 and m is an integer ranging from 1 to 8;
R³ denotes a C₁₅-C₂₆ hydrocarbon-based group, saturated or unsaturated in the alpha position, this group possibly being substituted with one or more C₁-C₁₄ alkyl groups; it being understood that in the case of natural ceramides or glycoceramides, R³ may also denote a C₁₅-C₂₆ alpha-hydroxyalkyl group, the hydroxyl group being optionally esterified with a C₁₆-C₃₀ alpha-hydroxy acid.

The ceramides that are more particularly preferred are the compounds for which R¹ denotes a saturated or unsaturated alkyl derived from C₁₆-C₂₂ fatty acids; R² denotes a hydrogen atom and R³ denotes a saturated linear C₁₅ group.

Preferentially, use is made of ceramides for which R¹ denotes a saturated or unsaturated alkyl group derived from C₁₄-C₃₀ fatty acids; R² denotes a galactosyl or sulfogalactosyl group; and R³ denotes a —CH═CH—(CH₂)₁₂—CH₃ group.

Use may also be made of the compounds for which R¹ denotes a saturated or unsaturated alkyl radical derived from C₁₂-C₂₂ fatty acids; R² denotes a galactosyl or sulfogalactosyl radical and R³ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

The non-silicone solid fatty substances are preferably chosen from solid fatty alcohols, solid fatty acid and/or fatty alcohol esters and mixtures thereof, preferentially from solid fatty alcohols and mixtures thereof, in particular from cetyl alcohol, stearyl alcohol and mixtures thereof such as cetylstearyl or cetearyl alcohol.

Butters may also be used.

For the purposes of the present invention, the term “butter” (also referred to as a “pasty fatty substance”) means a lipophilic fatty compound with a reversible solid/liquid change of state, including at a temperature of 25° C. and at atmospheric pressure (760 mmHg) a liquid fraction and a solid fraction. Preferably, the butter(s) according to the invention have a starting melting temperature above 25° C. and an end melting temperature below 60° C.

Preferably, the particular butter(s) are of plant origin, such as those described in Ullmann's Encyclopedia of Industrial Chemistry (“Fats and Fatty Oils”, A. Thomas, published online: JUN 15, 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter (Butyrospermum parkii), Borneo butter or fat or tengkawang tallow (Shorea stenoptera), shorea butter, illipè butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

An example of a preferred butter is shea butter.

In a known manner, shea butter is extracted from the fruit (also called “kernels” or “nuts”) of the Butyrospemumparkii tree. Each fruit contains between 45% and 55% fat, which is generally extracted and refined.

Advantageously, the composition according to the invention comprises at least one non-silicone fatty substance other than fatty amines, preferably chosen from solid fatty substances, liquid fatty substances, butters and mixtures thereof, more preferentially from solid fatty alcohols, solid fatty acid and/or fatty alcohol esters and plant oils, liquid fatty acid and/or fatty alcohol esters other than triglycerides, butters of plant origin and mixtures thereof, such as cetyl alcohol, stearyl alcohol and mixtures thereof, such as cetylstearyl alcohol, cetearyl alcohol and mixtures thereof, cetyl esters, isopropyl myristate, sunflower oil, avocado oil, shea butter, and mixtures thereof.

Preferably, the composition according to the invention comprises one or more liquid non-silicone fatty substances other than fatty amines, preferentially chosen from plant oils, liquid fatty acid and/or fatty alcohol esters other than triglycerides, and mixtures thereof.

Preferably, the composition according to the invention comprises one or more solid non-silicone fatty substances other than fatty amines, preferentially chosen from solid fatty alcohols, solid esters of fatty acids and/or fatty alcohols, and mixtures thereof.

Preferably, the composition according to the invention comprises one or more liquid non-silicone fatty substances other than fatty amines, preferentially chosen from plant oils, liquid fatty acid and/or fatty alcohol esters other than triglycerides, and mixtures thereof, and one or more solid non-silicone fatty substances other than fatty amines, preferentially chosen from solid fatty alcohols, solid fatty acid and/or fatty alcohol esters, and mixtures thereof.

Advantageously, the total content of the non-silicone fatty substance(s) other than fatty amines ranges from 0.1% to 35% by weight, preferably from 1% to 30% by weight, more preferentially from 2% to 25% by weight, better still from 5% to 20% by weight, even better still from 8% to 15% by weight relative to the total weight of the composition.

In a particular embodiment, when the composition according to the invention comprises one or more solid and/or liquid and/or pasty non-silicone fatty substance(s) other than fatty amines, their total content preferably ranges from 0.1% to 35% by weight, preferably from 1% to 30% by weight, more preferentially from 2% to 25% by weight, better still from 5% to 20% by weight, even better still from 8% to 15% by weight relative to the total weight of the composition.

### Cationic Polymer

The composition according to the invention may also comprise at least one cationic polymer preferably chosen from cationic polysaccharides.

The term “cationic polymer” means any polymer comprising cationic groups and/or groups that can be ionized to cationic groups. Preferably, the cationic polymer is hydrophilic or amphiphilic. The preferred cationic polymers are chosen from those that contain units including primary, secondary, tertiary and/or quaternary amine groups that may either form part of the main polymer chain or may be borne by a side substituent directly connected thereto.

The cationic polymers that may be used preferably have a weight-average molar mass (Mw) of between 500 and 5×10⁶ approximately and preferably between 10¹ and 3×10⁶ approximately.

Among the cationic polysaccharides, mention may be made of cationic celluloses and galactomannan gums. Mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

Cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example a chloride). Such products are notably sold under the names Jaguar C13 S, Jaguar C 15, Jaguar C 17 and Jaguar C162 by the company Rhodia.

According to a preferred embodiment, the cationic polymer(s) are preferably chosen from cationic polysaccharides, more preferentially chosen from cationic galactomannan gums, better still chosen from cationic guar gums.

Advantageously, when they are present, the total content of the cationic polymer(s) ranges from 0.0001% to 5% by weight, preferably from 0.001% to 3% by weight, more preferentially from 0.01% to 2% by weight, and better still from 0.05% to 1% by weight, relative to the total weight of the composition.

### Cationic Surfactant

The composition according to the invention may also comprise at least one cationic surfactant, other than the fatty amines described previously.

The term “cationic surfactant” means a surfactant that is positively charged when it is contained in the compositions according to the invention. This surfactant may bear one or more positive permanent charges or may contain one or more cationizable functions within the compositions according to the invention.

Preferably, the cationic surfactant(s) are chosen from quaternary ammonium salts and mixtures thereof.

Examples of quaternary ammonium salts that may notably be mentioned include:
those corresponding to the general formula (X) below:

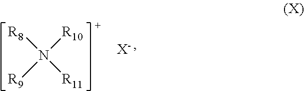

in which the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 carbon atoms and preferably from 12 to 24 carbon atoms. The aliphatic groups may include heteroatoms notably such as oxygen, nitrogen, sulfur and halogens.

The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups, X⁻ is an anion chosen from the group of halides, phosphates, acetates, lactates, (C₁-C₄)alkyl sulfates, and (C₁-C₄)alkylsulfonates or (C₁-C₄)alkylarylsulfonates.

Among the quaternary ammonium salts of formula (X), those that are preferred are, on the one hand, tetraalkylammonium chlorides, for instance dialkyldimethylammonium or alkyltrimethylammonium chlorides in which the alkyl group contains approximately from 12 to 22 carbon atoms, in particular behenyltrimethylammonium, distearyldimethylammonium, cetyltrimethylammonium or benzyldimethylstearylammonium chlorides, or, on the other hand, palmitylamidopropyltrimethylammonium chloride or the stearamidopropyldimethyl(myristyl acetate)ammonium chloride sold under the name Ceraphyl® 70 by the company Van Dyk;
quaternary ammonium salts of imidazoline, for instance those of formula (XI) below:

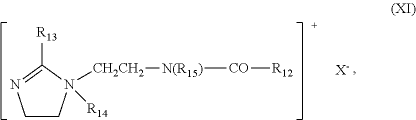

in which R₁₂ represents an alkenyl or alkyl group including from 8 to 30 carbon atoms, for example tallow fatty acid derivatives, R₁₃ represents a hydrogen atom, a C₁-C₄ alkyl group or an alkenyl or alkyl group including from 8 to 30 carbon atoms, R₁₄ represents a C₁-C₄ alkyl group, R₁₅ represents a hydrogen atom or a C₁-C₄ alkyl group, and X⁻ is an anion chosen from the group of halides, phosphates, acetates, lactates, (C₁-C₄)alkyl sulfates, and (C₁-C₄)alkylsulfonates or (C₁-C₄)alkylarylsulfonates.

Preferably, R₁₂ and R₁₃ denote a mixture of alkenyl or alkyl groups including from 12 to 21 carbon atoms, for example derived from tallow fatty acids, R₁₄ denotes a methyl group and R₁₅ denotes a hydrogen atom. Such a product is sold, for example, under the name Rewoquat® W 75 by the company Rewo;
quaternary diammonium or triammonium salts, in particular of formula (XII) below:

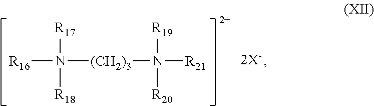

in which R₁₆ denotes an alkyl group including from about 16 to 30 carbon atoms, which is optionally hydroxylated and/or interrupted with one or more oxygen atoms; R₁₇ is chosen from hydrogen, an alkyl group including from 1 to 4 carbon atoms or a group —(CH₂)₃-N+(R₁₆ₐ)(R₁₇ₐ)(R₁₈ₐ), R₁₆ₐ, R₁₇ₐ, R₁₈ₐ, R₁₈, R₁₉, R₂₀ and R₂₁, which may be identical or different, are chosen from hydrogen and an alkyl group including from 1 to 4 carbon atoms, and X⁻ is an anion chosen from the group of halides, acetates, phosphates, nitrates, (C₁-C₄)alkyl sulfates, and (C₁-C₄)alkylsulfonates or (C₁-C₄)alkylarylsulfonates, in particular methyl sulfate and ethyl sulfate.

Such compounds are, for example, Finquat CT-P, sold by the company Finetex (Quaternium 89), and Finquat CT, sold by the company Finetex (Quaternium 75);
quaternary ammonium salts containing one or more ester functions, for instance those of formula (XIII) below:

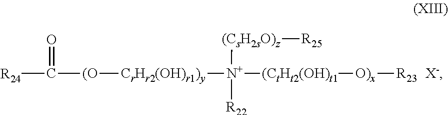

in which: R₂₂ is chosen from C₁-C₆ alkyl groups and C₁-C₆ hydroxyalkyl or dihydroxyalkyl groups; R₂₃ is chosen from: the group —C(O)R₂₆, linear or branched, saturated or unsaturated C₁-C₂₂ hydrocarbon-based groups R₂₇, a hydrogen atom; R₂₅ is chosen from: the group —C(O)R₂₈, linear or branched, saturated or unsaturated C₁-C₆ hydrocarbon-based groups R₂₉, a hydrogen atom; R₂₄, R₂₆ and R₂₈, which may be identical or different, are chosen from linear or branched, saturated or unsaturated C₇-C₂₁ hydrocarbon-based groups; r, s and t, which may be identical or different, are integers from 2 to 6; r1 and t1, which may be identical or different, are 0 or 1; r2+r1=2 r and t1+t2=2 t, y is an integer from 1 to 10, x and z, which may be identical or different, are integers from 0 to 10, X⁻ is a simple or complex, organic or inorganic anion, with the proviso that the sum x+y+z is from 1 to 15, that when x is 0 then R₂₃ denotes R₂₇ and that when z is 0 then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and contain from 12 to 22 carbon atoms, or may be short and contain from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate or a (C₁-C₄)alkylsulfonate or (C₁-C₄)alkylarylsulfonate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride, methyl sulfate or ethyl sulfate.

Use is made more particularly, in the composition according to the invention, of the ammonium salts of formula (XIII) in which: R₂₂ denotes a methyl or ethyl group, x and y are equal to 1, z is equal to 0 or 1, r, s and t are equal to 2; R₂₃ is chosen from: the group —C(O)R₂₆, methyl, ethyl or C₁₄-C₂₂ hydrocarbon-based groups, or a hydrogen atom, R₂₅ is chosen from: the group —C(O)R₂₈, or a hydrogen atom, R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (XIII), examples that may be mentioned include salts, notably the chloride or methyl sulfate, of diacyloxyethyldimethylammonium, diacyloxyethylhydroxyethylmethylammonium, monoacyloxyethyldihydroxyethylmethylammonium, triacyloxyethylmethyl-ammonium or monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by quaternization by means of an alkylating agent such as an alkyl halide, preferably a methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may also be made of the behenoylhydroxypropyltrimethylammonium chloride sold, for example, by the company Kao under the name Quartamin BTC 131.

Mention may also be made of distearoylethylhydroxyethylmethylammonium methosulfate, dipalmitoylethylhydroxyethylammonium methosulfate or distearoylethylhydroxyethylammonium methosulfate.

Among the quaternary ammonium salts containing at least one ester function that may be used, it is preferred to use dipalmitoylethylhydroxyethylmethylammonium salts, in particular dipalmitoylethylhydroxyethylammonium methosulfate.

Preferably, the ammonium salts containing at least one ester function contain two ester functions.

Advantageously, the cationic surfactant(s) are chosen from quaternary ammonium salts, preferably from those corresponding to the general formula (X), those corresponding to the general formula (XIII), and mixtures thereof.

Preferably, the quaternary ammonium salts corresponding to the general formula (X) are chosen from tetraalkylammonium chlorides, such as dialkyldimethylammonium or alkyltrimethylammonium chlorides in which the alkyl group comprises from 12 to 22 carbon atoms, in particular behenyltrimethylammonium, distearyldimethylammonium, cetyltrimethylammonium, benzyldimethylstearylammonium chlorides and mixtures thereof.

Examples of quaternary ammonium salts corresponding to the general formula (XIII) may be distearoylethylhydroxyethylmethylammonium methosulfate, dipalmitoylethylhydroxyethylammonium methosulfate, distearoylethylhydroxyethylammonium methosulfate, and mixtures thereof.

According to a particularly preferred embodiment, the quaternary ammonium salts are chosen from tetraalkylammonium chlorides, such as dialkyldimethylammonium or alkyltrimethylammonium chlorides in which the alkyl group comprises from 12 to 22 carbon atoms, in particular behenyltrimethylammonium, distearyldimethylammonium, cetyltrimethylammonium and benzyldimethylstearylammonium chlorides, dipalmitoylethylhydroxyethylmethylammonium salts, in particular dipalmitoylethylhydroxyethylammonium methosulfate, and mixtures thereof.

Advantageously, the total content of the cationic surfactant(s), other than the fatty amines described previously, ranges from 0.01% to 15% by weight, preferably from 0.1% to 10% by weight, more preferentially from 0.5% to 8% by weight, better still from 1% to 6% by weight relative to the total weight of the composition.

### Silicone

The composition according to the invention may also comprise at least one silicone, which may be chosen from non-amino silicones, amino silicones and mixtures thereof.

The silicone(s) are preferably chosen from amino silicone(s).

The composition according to the invention may thus comprise one or more non-amino silicones, which may be solid or liquid, preferably liquid (at 25° C., 1 atm), and volatile or non-volatile.

The non-amino silicones that may be used may be soluble or insoluble in the composition according to the invention; they may be in oil, wax, resin or gum form; silicone oils and gums are preferred.

Silicones are notably described in detail in Walter Noll's Chemistry and Technology of Silicones (1968), Academic Press.

The volatile silicones may be chosen from those with a boiling point of between 60° C. and 260° C. (at atmospheric pressure) and in particular from:
i) cyclic polydialkylsiloxanes including from 3 to 7 and preferably 4 to 5 silicon atoms, such as
   octamethylcyclotetrasiloxane (D4) and decamethylcyclopentasiloxane (D5). Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia;
   cyclocopolymers of the dimethylsiloxane/methylalkylsiloxane type having the chemical structure:

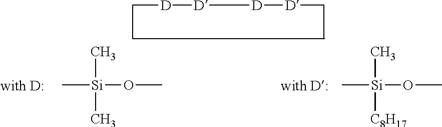

Mention may be made of Volatile Silicone FZ 3109 sold by the company Union Carbide.
mixtures of cyclic silicones with silicon-based organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;
ii) linear polydialkylsiloxanes containing 2 to 9 silicon atoms, which generally have a viscosity of less than or equal to 5×10⁻⁶ m²/s at 25° C., such as decamethyltetrasiloxane.

Other silicones belonging to this category are described in the article published in Cosmetics and Toiletries, Vol. 91, January 76, pages 27-32—Todd & Byers Volatile silicone fluids for cosmetics; mention may be made of the product sold under the name SH 200 by the company Toray Silicone.

Among the non-volatile silicones, mention may be made, alone or as a mixture, of polydialkylsiloxanes and notably polydimethylsiloxanes (PDMS or dimethicone), polydiarylsiloxanes, polyalkylarylsiloxanes, silicone gums and resins, and also non-amino organopolysiloxanes (or organomodified polysiloxanes, or alternatively organomodified silicones) which are polysiloxanes including in their structure one or more non-amino organofunctional groups, generally attached via a hydrocarbon-based group, and preferably chosen from aryl groups, alkoxy groups and polyoxyethylene and/or polyoxypropylene groups.

The organomodified silicones may be polydiarylsiloxanes, notably polydiphenylsiloxanes, and polyalkylarylsiloxanes functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes including:
polyoxyethylene and/or polyoxypropylene groups optionally including C₆-C₂₄ alkyl groups, such as dimethicone copolyols, and notably those sold by the company Dow Corning under the name DC 1248 or the oils Silwet® L 722, L 7500, L 77 and L 711 from the company Union Carbide; or alternatively (C₁₂)alkylmethicone copolyols, and notably those sold by the company Dow Corning under the name Q2-5200;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by the company Goldschmidt;
hydroxylated groups, for instance polyorganosiloxanes bearing a hydroxyalkyl function;
acyloxyalkyl groups, such as the polyorganosiloxanes described in patent U.S. Pat. No. 4,957,732;
anionic groups of the carboxylic acid type, as described, for example, in EP 186 507, or of the alkylcarboxylic type, such as the product X-22-3701E from the company Shin-Etsu; or else of the 2-hydroxyalkylsulfonate or 2-hydroxyalkylthiosulfate type, such as the products sold by the company Goldschmidt under the names Abil® S201 and Abil® S255;

The silicones may also be chosen from polydialkylsiloxanes, among which mention may be made mainly of polydimethylsiloxanes bearing trimethylsilyl end groups (CTFA: dimethicone). Among these polydialkylsiloxanes, mention may be made of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups (CTFA: dimethiconol), such as the oils of the 48 series from the company Rhodia.

In this category of polydialkylsiloxanes, mention may also be made of the products sold under the names Abil Wax® 9800 and 9801 by the company Goldschmidt, which are poly(C₁-C₂₀)dialkylsiloxanes.

Products that may be used more particularly in accordance with the invention are mixtures such as:
mixtures formed from a polydimethylsiloxane with a hydroxy-terminated chain, or dimethiconol (CTFA), and from a cyclic polydimethylsiloxane, also known as cyclomethicone (CTFA), such as the product Q2-1401 sold by the company Dow Corning.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

The non-amino silicones that are more particularly preferred according to the invention are polydimethylsiloxanes containing trimethylsilyl end groups (CTFA: dimethicone).

The composition according to the invention may comprise one or more amino silicones.

The term “amino silicone” denotes any silicone including at least one primary, secondary or tertiary amine or a quaternary ammonium group.

The amino silicones that may be used according to the present invention may be volatile or non-volatile and cyclic, linear or branched, and preferably have a viscosity ranging from 5×10⁻⁶ to 2.5 m²/s at 25° C., for example from 1×10⁻⁵ to 1 m²/s.

Preferably, the amino silicone(s) are chosen, alone or as mixtures, from the following compounds:
a) the polysiloxanes corresponding to formula (I):

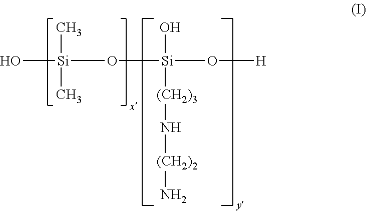

in which x′ and y′ are integers such that the weight-average molecular mass (Mw) is between 5000 and 500 000 g/mol;
b) the amino silicones corresponding to formula (II):

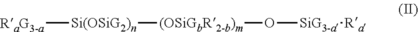

in which:
G, which may be identical or different, denotes a hydrogen atom or a phenyl, OH, C₁-C₈ alkyl, for example methyl, or C₁-C₈ alkoxy, for example methoxy, group;
a and a′, which may be identical or different, denote 0 or an integer from 1 to 3, in particular 0, with the proviso that at least one from among a and a′ is equal to zero,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and —R′, which may be identical or different, denotes a monovalent radical of formula —C_{q}H_{2q}L in which q is a number ranging from 2 to 8 and L is an optionally quaternized amine group chosen from the following groups: —NR″-Q-N(R″)₂, —N(R″)₂, —N+(R″)₃ A⁻, —N+H(R″)₂ A⁻, —N+H₂(R″) A⁻, —NR″-Q-N+(R″)H₂ A⁻, —NR″-Q-N+(R″)₂H A⁻ and —NR″-Q-N+(R″)₃ A⁻, in which R″, which may be identical or different, denotes hydrogen, phenyl, benzyl or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, notably a halide, such as fluoride, chloride, bromide or iodide.

Preferably, the amino silicones of formula (II) may be chosen from:

### (i) the “Trimethylsilyl Amodimethicone” Silicones Corresponding to Formula (III):

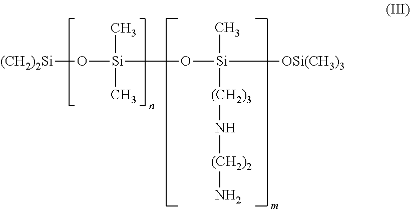

in which m and n are numbers such that the sum (n+m) varies from 1 to 2000, preferably from 20 to 1000, in particular from 50 to 600, better still from 50 to 150; n possibly denoting a number from 0 to 1999 and notably from 49 to 149 and m possibly denoting a number from 1 to 2000 and notably from 1 to 10;

### (ii) the Silicones of Formula (IV) Below:

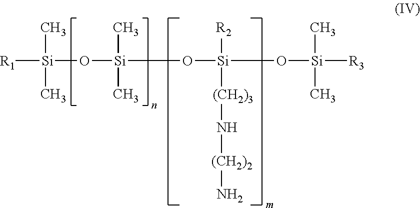

in which:
   m and n are numbers such that the sum (n+m) ranges from 1 to 1000, in particular from 50 to 250 and more particularly from 100 to 200; n denoting a number from 0 to 999 and notably from 49 to 249 and more particularly from 125 to 175, and m denoting a number from 1 to 1000, notably from 1 to 10 and more particularly from 1 to 5; and
   R₁, R₂ and R₃, which may be identical or different, represent a hydroxyl or C₁-C₄ alkoxy radical, at least one of the radicals R₁ to R₃ denoting an alkoxy radical.
      Preferably, the alkoxy radical is a methoxy radical.
      The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 g/mol and more particularly from 3500 to 200 000 g/mol.

(iii) the Silicones of Formula (V) Below:

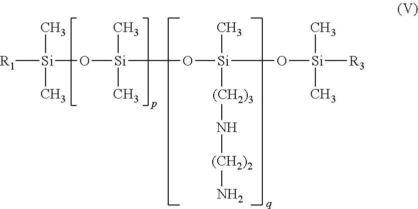

in which:
   p and q are numbers such that the sum (p+q) ranges from 1 to 1000, in particular from 50 to 350 and more particularly from 150 to 250; p denoting a number from 0 to 999, notably from 49 to 349 and more particularly from 159 to 239, and q denoting a number from 1 to 1000, notably from 1 to 10 and more particularly from 1 to 5; and
   R₁ and R₂, which are different, represent a hydroxyl or C₁-C₄ alkoxy radical, at least one of the radicals R₁ or R₂ denoting an alkoxy radical.
      Preferably, the alkoxy radical is a methoxy radical.
      The hydroxy/alkoxy mole ratio generally ranges from 1:0.8 to 1:1.1 and preferably from 1:0.9 to 1:1 and more particularly is equal to 1:0.95.
      The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) may include in their composition one or more other amino silicones, the structure of which is different from formula (IV) or (V). A product containing amino silicones of structure (IV) is sold by the company Wacker under the name Belsil® ADM 652. A product containing amino silicones of structure (V) is sold by Wacker under the name Fluid WR 1300®. Another product containing amino silicones of structure (XIV) is sold by Wacker under the name Belsil ADM LOG 1®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nm. Preferably, notably as amino silicones of formula (V), use is made of microemulsions of which the mean particle size ranges from 5 nm to 60 nm (limits included) and more particularly from 10 nm to 50 nm (limits included). Thus, use may be made according to the invention of the amino silicone microemulsions of formula (V) sold under the names Finish CT 96 E® or SLM 28020® by the company Wacker.

### (iv) the Silicones of Formula (VI) Below:

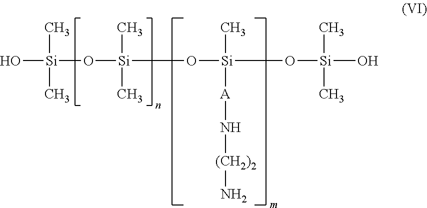

in which:
m and n are numbers such that the sum (n+m) ranges from 1 to 2000 and in particular from 50 to 150, n denoting a number from 0 to 1999 and notably from 49 to 149, and m denoting a number from 1 to 2000 and notably from 1 to 10; and
A denotes a linear or branched, preferably linear, alkylene radical containing from 4 to 8 carbon atoms and preferably 4 carbon atoms.

The weight-average molecular mass (Mw) of these amino silicones preferably ranges from 2000 to 1 000 000 g/mol and more particularly from 3500 to 200 000 g/mol.

A silicone corresponding to this formula is, for example, Xiameter MEM 8299 Emulsion from Dow Corning.

### (v) the Silicones of Formula (VII) Below:

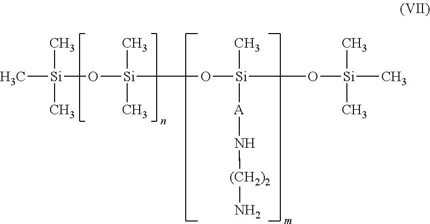

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
   A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1 000 000 g/mol and more particularly from 1000 to 200 000 g/mol.

A silicone corresponding to this formula is, for example, DC2-8566 Amino Fluid from Dow Corning;
c) the amino silicones corresponding to formula (VIII):

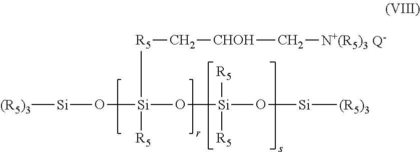

in which:
   R₅ represents a monovalent hydrocarbon-based radical containing from 1 to 18 carbon atoms, and in particular a C₁-C₁₈ alkyl or C₂-C₁₈ alkenyl radical, for example methyl;
   R₆ represents a divalent hydrocarbon-based radical, notably a C₁-C₁₈ alkylene radical or a divalent C₁-C₁₈, for example C₁-C₈, alkyleneoxy radical linked to the Si via an SiC bond;
   Q⁻ is an anion such as a halide ion, notably chloride, or an organic acid salt, notably acetate;
   r represents a mean statistical value ranging from 2 to 20 and in particular from 2 to 8;
and
   s represents a mean statistical value ranging from 20 to 200 and in particular from 20 to 50.
d) the quaternary ammonium silicones of formula (IX):

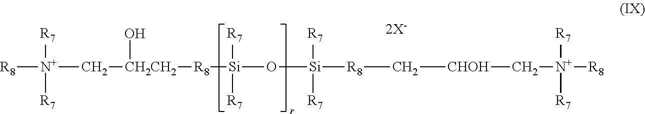

in which:
   R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
   R₆ represents a divalent hydrocarbon-based radical, notably a C₁-Cis alkylene radical or a divalent C₁-C₁₈, for example C₁-C₈, alkyleneoxy radical linked to the Si via an SiC bond;
   R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
   X⁻ is an anion such as a halide ion, notably chloride, or an organic acid salt, notably acetate; and
   r represents a mean statistical value ranging from 2 to 200 and in particular from 5 to 100.

These silicones are for example described in patent application EP-A-0 530 974; mention may in particular be made of the silicone having the INCI name: Quaternium 80.

Silicones falling within this category are the silicones sold by the company Goldschmidt under the names Abil Quat 3270, Abil Quat 3272 and Abil Quat 3474;
e) the amino silicones of formula (X):

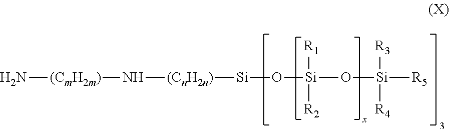

in which:
   R₁, R₂, R₃ and R₄, which may be identical or different, denote a C₁-C₄ alkyl radical or a phenyl group,
   R₅ denotes a C₁-C₄ alkyl radical or a hydroxyl group,
   n is an integer ranging from 1 to 5,
   m is an integer ranging from 1 to 5, and
   x is chosen such that the amine number ranges from 0.01 to 1 meq/g;
f) multiblock polyoxyalkylene amino silicones, of the type (AB)ₙ, A being a polysiloxane block and B being a polyoxyalkylene block including at least one amine group.

Said silicones are preferably formed from repeating units having the following general formulae:

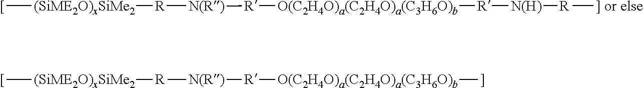

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C₂-C₁₂ hydrocarbon-based radical, optionally including one or more heteroatoms such as oxygen; preferably, R, which may be identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which may be identical or different, represent a linear or branched divalent C₂-C₁₂ hydrocarbon-based radical, optionally including one or more heteroatoms such as oxygen; preferably, R′, which may be identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 g/mol and more particularly between 10 000 and 200 000 g/mol.

Mention may notably be made of the silicones sold under the name Silsoft A-843 or Silsoft A+by Momentive.
g) the amino silicones of formulae (XI) and (XII):

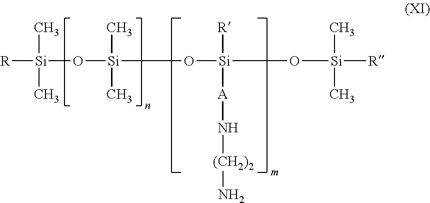

in which:
   R, R′ and R″, which may be identical or different, denote a C₁-C₄ alkyl group or a hydroxyl group,
   A denotes a C₃ alkylene radical; and
   m and n are numbers such that the weight-average molecular mass of the compound is between 5000 and 500 000;

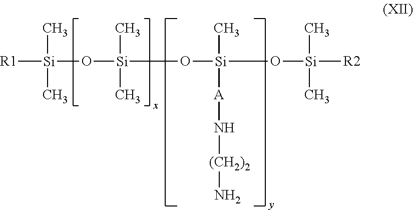

in which:
   x and y are numbers ranging from 1 to 5000; preferably, x ranges from 10 to 2000 and more preferentially from 100 to 1000; preferably, y ranges from 1 to 100;
   R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.

Preferably, A comprises from 3 to 6 carbon atoms, more preferentially 4 carbon atoms; preferably, A is branched. Mention may be made in particular of the following divalent groups: —CH₂CH₂CH₂— and —CH₂CH(CH₃)CH₂—.

Preferably, R₁ and R₂ are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which may be identical or different, are chosen from hexadecyl (cetyl) and octadecyl (stearyl) groups.

Preferably, in the silicone of formula (XII):
x ranges from 10 to 2000 and in particular from 100 to 1000;
y ranges from 1 to 100;
A comprises from 3 to 6 carbon atoms and notably 4 carbon atoms;
preferably, A is branched; more particularly, A is chosen from the following divalent groups: —CH₂CH₂CH₂ and —CH₂CH(CH₃)CH₂—; and
R₁ and R₂ independently are saturated linear alkyl groups comprising from 6 to 30 carbon atoms, preferably from 8 to 24 carbon atoms and in particular from 12 to 20 carbon atoms; notably chosen from dodecyl, tetradecyl, pentadecyl, hexadecyl, heptadecyl, octadecyl, nonadecyl and eicosyl groups; preferentially, R₁ and R₂, which may be identical or different, are chosen from hexadecyl (cetyl) and octadecyl (stearyl) groups.

A silicone of formula (XII) that is preferred is bis-cetearyl amodimethicone. Mention may be made in particular of the amino silicone sold under the name Silsoft AX by Momentive.
h) polysiloxanes and notably polydimethylsiloxanes, including primary amine groups at only one chain end or on side chains, such as those of formula (XIV), (XV) or (XVI):

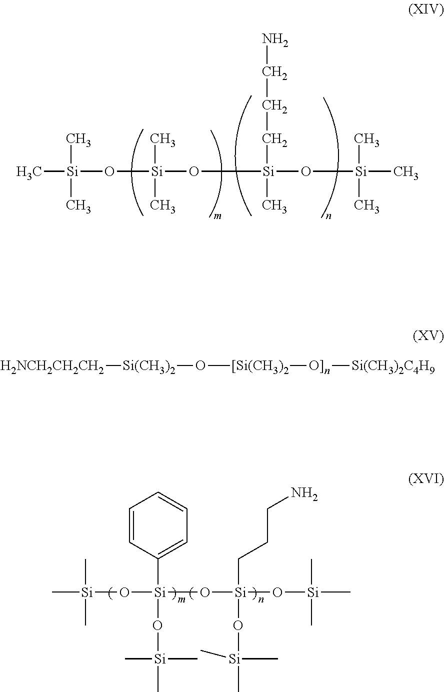

In formula (XIV), the values of n and m are such that the weight-average molecular mass of the amino silicone is between 1000 and 55 000.

As examples of amino silicones of formula (XIV), mention may be made of the products sold under the names AMS-132, AMS-152, AMS-162, AMS-163, AMS-191 and AMS-1203 by the company Gelest and KF-8015 by the company Shin-Etsu.

In formula (XV), the value of n is such that the weight-average molecular mass of the amino silicone is between 500 and 3000.

As examples of amino silicones of formula (XV), mention may be made of the products sold under the names MCR-A1 1 and MCR-A12 by the company Gelest.

In formula (XVI), the values of n and m are such that the weight-average molecular mass of the amino silicone is between 500 and 50 000.

As examples of amino silicones of formula (XVI), mention may be made of the aminopropyl phenyl trimethicone sold under the name DC 2-2078 Fluid by the company Dow Corning.

The cosmetic composition according to the invention may also comprise, as silicone, an amino silicone corresponding to formula (XVIII) below:

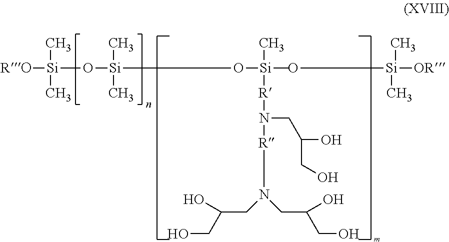

in which:
n is a number between 1 and 1000, preferably between 10 and 500, better still between 25 and 100, even better still between 50 and 80;
m is a number between 1 and 200, preferably between 1 and 100, better still between 1 and 10 and even better still between 1 and 5;
R″′, which may be identical or different, preferably identical, are saturated or unsaturated, linear or branched alkyl radicals comprising from 8 to 30 carbon atoms, preferably from 10 to 24 carbon atoms, notably from 12 to 18 carbon atoms; said radicals possibly being substituted with one or more hydroxyl OH groups;
R′ is a linear or branched divalent alkylene radical containing from 1 to 6 carbon atoms, notably from 2 to 5 carbon atoms;
R″ is a linear or branched divalent alkylene radical containing from 1 to 6 carbon atoms, notably from 1 to 5 carbon atoms.

Preferably, the radicals R″′, which may be identical or different, are saturated linear alkyl radicals comprising from 8 to 30 carbon atoms, preferably from 10 to 24 carbon atoms, notably from 12 to 18 carbon atoms; mention may be made in particular of dodecyl, tridecyl, tetradecyl, pentadecyl, hexadecyl, heptadecyl and octadecyl radicals; preferentially, the radicals R″′, which may be identical or different, are chosen from saturated linear alkyl radicals containing from 12 to 16 carbon atoms, which are notably C₁₃, C₁₄ or C₁₅ radicals, alone or as a mixture, and better still represent a mixture of C₁₃, C₁₄ and C₁₅ radicals.

Preferably, the radicals R″′ are identical.

Preferably, R′ is a linear or branched, preferably branched, divalent alkylene radical comprising from 1 to 6 carbon atoms, notably from 2 to 5 carbon atoms; notably a —CH₂—CH₂—CH₂—, —CH₂—CH(CH₃)—CH₂— or —CH₂—CH₂—CH(CH₃)— radical.

Preferably, R″ is a linear divalent alkylene radical comprising from 1 to 6 carbon atoms, notably from 1 to 4 carbon atoms, in particular a —CH₂—CH₂— radical.

Advantageously, when the composition according to the invention comprises at least one silicone chosen from amino silicones, said amino silicone is preferably chosen from the silicones of formula (II), more preferentially from the silicones of formula (VI) and mixtures thereof.

When the composition comprises at least one silicone, the total content of the silicone(s) is preferably from 0.01% to 15% by weight, preferably from 0.05% to 10% by weight, and better still from 0.5% to 5% by weight relative to the total weight of the composition.

When the composition comprises at least one amino silicone, the total content of the amino silicone(s) preferably ranges from 0.01% to 15% by weight, more preferentially from 0.05% to 10% by weight, and better still from 0.5% to 5% by weight, relative to the total weight of the composition.

### Non-Cationic Polysaccharide

The composition according to the invention may also comprise at least one non-cationic polysaccharide. In other words, said non-cationic polysaccharide is different from the cationic polymers described previously. Preferably, said non-cationic polysaccharide(s) are chosen from nonionic polysaccharides.

The nonionic polysaccharides are preferably chosen, alone or as a mixture, from celluloses, starches, galactomannans and nonionic derivatives thereof, notably ethers or esters thereof.

These polymers may be physically or chemically modified. Mention may be made, as physical treatment, of the temperature and mention may be made, as chemical treatment, of esterification, etherification, amidation and oxidation reactions, in so far as these treatments make it possible to give polymers that are nonionic.

As galactomannans that may be used, mention may be made of nonionic guar gums which can be modified with (poly)hydroxy(C₁-C₆)alkyl groups, notably hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are, for example, sold under the trade names Jaguar HP8, Jaguar HP60, Jaguar HP120, Jaguar HIP105 SGI and Jaguar HP8 SGI by the company Rhodia Chimie.

The botanical origin of the starch molecules that may be used in the present invention may be cereals or tubers. Thus, the starches are chosen, for example, from corn starch, rice starch, cassava starch, barley starch, potato starch, wheat starch, sorghum starch and pea starch. The starches may be chemically or physically modified, notably by one or more of the following reactions: pregelatinization, oxidation, crosslinking, esterification, etherification, amidation, heat treatments.

The starch molecules may be derived from any plant source of starch, notably such as corn, potato, oat, rice, tapioca, sorghum, barley or wheat. It is also possible to use hydrolysates of the starches mentioned above. The starch is preferably derived from potato.

The nonionic polysaccharides may also be cellulose-based polymers not including a C10-C30 fatty chain in their structure.

According to the invention, the term “cellulose-based” polymer refers to any polysaccharide compound bearing in its structure sequences of glucose residues linked together by β-1,4 bonds; the cellulose-based polymers may be unsubstituted celluloses, and/or derivatives of nonionic celluloses.

Thus, the cellulose-based polymers that may be used according to the invention may be chosen from unsubstituted celluloses, including those in a microcrystalline form, and cellulose ethers. Among these cellulose-based polymers, cellulose ethers, cellulose esters and cellulose ethers-esters are distinguished.

Among the nonionic cellulose ethers that may be mentioned are (C₁-C₄)alkylcelluloses, such as methylcelluloses and ethylcelluloses (for example Ethocel Standard 100 Premium from Dow Chemical); (poly)hydroxy(C₁-C₄)alkylcelluloses, such as hydroxymethylcelluloses, hydroxyethylcelluloses (for example Natrosol 250 HHR sold by Aqualon) and hydroxypropylcelluloses (for example Klucel EF from Aqualon); mixed (poly)hydroxy(C₁-C₄)alkyl(C₁-C₄)alkylcelluloses, such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Preferably, the nonionic polysaccharides are chosen, alone or as a mixture, from celluloses, galactomannans and nonionic derivatives thereof, notably ethers thereof, and better still, alone or as a mixture, from nonionic guar gums optionally modified with (poly)hydroxy(C1-C6)alkyl, in particular hydroxypropyl, groups; and/or celluloses, which may be substituted or unsubstituted, and cellulose ethers, such as (C1-C4)alkylcelluloses and (poly)hydroxy(C1-C4)alkylcelluloses such as hydroxyethylcellulose.

Preferentially, the nonionic polysaccharides are chosen from cellulose ethers such as (C1-C4)alkylcelluloses, and (poly)hydroxy(C1-C4)alkylcelluloses such as hydroxyethylcellulose, and mixtures thereof.

When the composition comprises at least one non-cationic polysaccharide, the total content of the non-cationic polysaccharide(s) advantageously ranges from 0.01% to 10% by weight, preferably from 0.05% to 5% by weight, more preferentially from 0.1% to 2% by weight, relative to the total weight of the composition.

The composition according to the invention is preferably aqueous, the water content ranging preferably from 50% to 95% by weight, more preferentially from 60% to 94% by weight, even more preferentially from 75% to 93% by weight, better still from 80% to 92% by weight, relative to the total weight of the composition.

When the composition is aqueous, the pH may range from 2 to 7, preferably from 3 to 5.

### Additives

The composition according to the invention may contain any adjuvant or additive normally used, other than the compounds as described previously.

Among the additives that may be contained in the composition according to the invention, mention may be made of anionic, nonionic or amphoteric polymers or mixtures thereof other than the polysaccharides described previously, antidandruff agents, anti-seborrhoeic agents, agents for combating hair loss and/or for promoting hair regrowth, vitamins and pro-vitamins including panthenol, sunscreens, mineral or organic pigments, plasticizers, solubilizers, opacifying or nacreous agents, antioxidants, hydroxy acids, fragrances and preserving agents.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the ready-to-use composition.

### Process

The present invention also relates to a process for dyeing keratin fibres, preferably the hair, which comprises the application to said keratin fibres of the composition according to the invention as defined previously.

The composition may be applied to wet or dry keratin fibres. On conclusion of the treatment, the keratin fibres are optionally rinsed with water, optionally washed with a shampoo and then rinsed with water, before being dried or left to dry.

Preferably, on conclusion of the treatment, the keratin fibres are rinsed, preferably without being washed after this rinsing step.

Preferably, the composition according to the invention is applied at a temperature of between 10° C. and 100° C., better still between 15° C. and 40° C.

The composition may then be left in place for a period usually ranging from 1 minute to 2 hours, preferably from 5 minutes to 1 hour 30 minutes, better still from 10 minutes to 1 hour.

Advantageously, after application of the dye composition, the hair may be subjected to a heat treatment. In practice, this operation may be performed using a hairstyling hood, a hairdryer, an infrared ray emitter or other standard heating appliances.

The temperature during the process is conventionally between room temperature (between 15 and 25° C.) and 80° C. and preferably between room temperature and 60° C.

After the treatment, the human keratin fibres are optionally rinsed with water, optionally washed with a shampoo and then rinsed with water, before being dried (mechanically with a towel or paper towel, or with heat) or left to dry.

Finally, the present invention relates to the use of the composition according to the invention as described above, for dyeing keratin fibres, and in particular the hair.

The present invention will now be described more specifically by means of examples, which do not in any way limit the scope of the invention. However, the examples make it possible to support specific features, variants and preferred embodiments of the invention.

### EXAMPLES

In the examples that follow, all the amounts are given as mass percentages of active material (AMN) relative to the total weight of the composition (unless otherwise mentioned).

### Example 1

Compositions A1 and A2, as described in Table 2 below, were prepared.

TABLE 2

| | A1 | A2 |
|---|---|---|
| Compositions | (invention) | (comparative) |
| Benzyl alcohol | 0.3 | 0.3 |
| Isopropyl myristate | 2.0 | 2.0 |
| Cetyl esters ⁽¹⁾ | 1.5 | 1.5 |
| Basic Yellow 87 | 0.03 | 0.03 |
| Basic Brown 17 | 0.12 | 0.12 |
| Basic Red 51 | 0.004 | 0.004 |
| Basic Orange 31 | 0.003 | 0.003 |
| Cetearyl alcohol | 6.0 | 6.0 |
| Butyrospermum parkii butter | 1.0 | 1.0 |
| Stearamidopropyldimethylamine | 2.4 | 2.4 |
| Sunflower (Helianthus annuus) oil | 1.0 | 1.0 |
| Avocado (Persea gratissima) oil | 0.01 | 0.01 |
| Tartaric acid | 0.42 | 0.42 |
| Tetrasodium glutamate diacetate | 0.24 | — |
| Preserving agents | qs | qs |
| Water | qs 100 | qs 100 |

⁽¹⁾ sold by the company Croda under the trade name SP Crodamol MS MBAL-PA-(SG)

### Protocol

Each of the compositions A1 and A2 was applied to locks of bleached, in a proportion of 5 g of composition per gram of lock. The locks were then placed on a hotplate set at 27° C. for 10 minutes. The locks were then rinsed and dried.

### Results

The colorimetric measurements were performed using a Konica Minolta 3600 spectrocolorimeter (illuminant D65, angle 10°, specular component included) in the CIELab system.

The chromaticity is calculated according to the equation:

$${C}^{⋆} = \sqrt{{( {a}^{⋆} )}^{2} + {( {b}^{⋆} )}^{2}}$$

The chromaticity is measured by the values a* and b*, a* representing the red/green axis and b* the yellow/blue axis.

The higher the chromaticity value C*, the more chromatic the colour of the treated keratin fibres.

The results are collated in Table 3 below.

TABLE 3

| | | A2 |
|---|---|---|
| Compositions | A1 (invention) | (comparative) |
| a* | 41.45 | 37.32 |
| b* | 43.40 | 42.02 |
| C* | 60.01 | 56.20 |

The locks ofhairtreated with composition A1 accordingto the invention have a higher C* value than those treated with the comparative composition A2. In other words, composition A1 according to the invention affords locks of hair with improved chromaticity relative to those treated with the comparative composition A2.

### Example 2

Compositions A1 and A3, as described in Table 4 below, were prepared.

TABLE 4

| Compositions | A1 | A3 |
|---|---|---|
| Benzyl alcohol | 0.3 | 0.3 |
| Isopropyl myristate | 2.0 | 2.0 |
| Cetyl esters ⁽¹⁾ | 1.5 | 1.5 |
| Basic Yellow 87 | 0.03 | 0.03 |
| Basic Brown 17 | 0.12 | 0.12 |
| Basic Red 51 | 0.004 | 0.004 |
| Basic Orange 31 | 0.003 | 0.003 |
| Cetearyl alcohol | 6.0 | 6.0 |
| Butyrospermum parkii butter | 1.0 | 1.0 |
| Stearamidopropyldimethylamine | 2.4 | — |
| PEG-2 oleamine | — | 2.4 |
| Sunflower (Helianthus annuus) oil | 1.0 | 1.0 |
| Avocado (Persea gratissima) oil | 0.01 | 0.01 |
| Tartaric acid | 0.42 | 0.42 |
| Tetrasodium glutamate diacetate | 0.24 | 0.24 |
| Preserving agents | qs | qs |
| Water | qs 100 | qs 100 |

### Protocol

Each of the compositions A1 and A3 was applied to locks of natural hair containing 90% white hair strands and to locks of permanent-waved hair containing 90% white hair strands, in a proportion of 5 g of composition per gram of lock. The locks were then placed on a hotplate set at 37° C. for 10 minutes. The locks were then rinsed and dried.

### Results: Dyeing Performance

The colorimetric measurements were performed using a Minolta CM3600D spectrophotometer (illuminant D65, angle 10°, specular component included) in the CIELab system.

Selectivity is represented by the colour difference ΔE between natural (NW) and permanent-waved (PW) coloured locks. The lower the value of ΔE, the lower the selectivity, and therefore the better the selectivity.

The results are collated in Table 5 below.

TABLE 5

| Compositions | Type of hair | L* | a* | b* | ΔE |
|---|---|---|---|---|---|
| A1 | NW | 46.1 | 17.2 | 27.3 | 7.03 |
| | PW | 46.0 | 22.5 | 31.9 | |
| A3 | NW | 45.4 | 16.4 | 25.6 | 12.26 |
| | PW | 48.1 | 25.0 | 33.9 | |

Composition A1 leads to a lower ΔE value, and therefore better selectivity, in comparison with composition A3.

### Results: Working Quality and Rheologv

The viscosity of the two compositions A1 and A3 was measured after 30 seconds, using a RHEOMAT Mettler RM 180 at 200 rpm with mobile number 4, and at a temperature of 25° C.

The results are collated in Table 6 below.

TABLE 6

| Compositions | A1 | A3 |
|---|---|---|
| Viscosity (Cps) | 4790 | 500 |

The viscosity of the composition A1 is of 4790 Cps, i.e. 4.79 Pa·s, while the viscosity of the composition A3 is of 500 Cps, i.e. 0.50 Pa·s.

The composition A3 is liquid, lumpy and runs the risk of dripping.

The composition A1 is homogeneous and unctuous, with a higher viscosity, and the product distributes well without risk of dripping.

Therefore, the composition according to the invention has even better properties when it also comprises at least one fatty amine which is not (poly)oxyalkylenated.

1-16. (canceled)

17. A cosmetic composition comprising:
(i) at least one compound chosen from N,N-dicarboxymethylglutamic acid, salts thereof, solvates thereof, solvates of the salts thereof, or mixtures thereof;
(ii) at least one coloring agent chosen from oxidation dyes, direct dyes, or mixtures thereof; and
(iii) at least one compound chosen from fatty amines, salts thereof, or mixtures thereof.

18. The composition of claim 17, wherein the (i) at least one compound chosen from N,N-dicarboxymethylglutamic acid, salts thereof, solvates thereof, solvates of the salts thereof, or mixtures thereof is chosen from alkali metal salts.

19. The composition of claim 17, wherein the (i) at least one compound chosen from N,N-dicarboxymethylglutamic acid, salts thereof, solvates thereof, solvates of the salts thereof, or mixtures thereof is tetrasodium glutamate diacetate.

20. The composition of claim 17, wherein the total amount of the (i) at least one compound chosen from N,N-dicarboxymethylglutamic acid, salts thereof, solvates thereof, solvates of the salts thereof, or mixtures thereof ranges from 0.001% to 15% by weight, relative to the total weight of the composition.

21. The composition of claim 17, wherein the (ii) at least one coloring agent is chosen from direct dyes.

22. The composition of claim 17, wherein the (ii) at least one coloring agent is chosen from azo direct dyes, hydrazono direct dyes, nitroaryl direct dyes, triarylmethane direct dyes, quinone direct dyes, or mixtures thereof.

23. The composition of claim 17, wherein the total amount of the (ii) at least one coloring agent ranges from 0.001% to 20% by weight, relative to the total weight of the composition.

24. The composition of claim 17, wherein the (iii) at least one compound chosen from fatty amines, salts thereof, or mixtures thereof is chosen from amidoamines of formula (A): 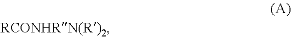
wherein:
R represents a substituted or unsubstituted, linear or branched, saturated or unsaturated monovalent hydrocarbon-based radical comprising from 5 to 29 carbon atoms;
R″ represents a divalent hydrocarbon-based radical comprising less than 6 carbon atoms; and
R′, which may be identical or different, each represents a substituted or unsubstituted, saturated or unsaturated, linear or branched, monovalent hydrocarbon-based radical comprising less than 6 carbon atoms; or
mixtures thereof.

25. The composition of claim 17, wherein the (iii) at least one compound chosen from fatty amines, salts thereof, or mixtures thereof is chosen from brassicamidopropyldimethylamine, stearamidopropyldimethylamine, behenamidopropyldimethylamine, oleamidopropyldimethylamine, or mixtures thereof.

26. The composition of claim 17, wherein the total amount of the (iii) at least one compound chosen from fatty amines, salts thereof, or mixtures thereof ranges from 0.01% to 6% by weight, relative to the total weight of the composition.

27. The composition of claim 17, further comprising: (iv) at least one non-silicone fatty substance other than fatty amines, wherein the (iv) at least one non-silicone fatty substance is chosen from solid fatty substances, liquid fatty substances, butters, or and mixtures thereof.

28. The composition of claim 27, wherein the (iv) at least one non-silicone fatty substance is chosen from solid fatty alcohols, solid fatty acid and/or fatty alcohol esters, plant oils, liquid fatty acid and/or fatty alcohol esters other than triglycerides, butters of plant origin, or mixtures thereof.

29. The composition of claim 27, wherein the total amount of the (iv) at least one non-silicone fatty substances other than fatty amines ranges from 0.1% to 35% by weight, relative to the total weight of the composition.

30. The composition of claim 17, further comprising: (v) at least one cationic polymer, wherein the (v) at least on cationic polymer is chosen from cationic polysaccharides.

31. The composition of claim 30, wherein the (v) at least on cationic polymer is chosen from cationic galactomannan gums.

32. The composition of claim 17, further comprising: (vi) at least one cationic surfactant other than the (iv) at least one compound chosen from fatty amines, salts thereof, or mixtures thereof, wherein the (vi) at least one cationic surfactant is chosen from quaternary ammonium salts.

33. The composition of claim 32, wherein the (vi) at least one cationic surfactant is chosen from:
quaternary ammonium salts of formula (X): 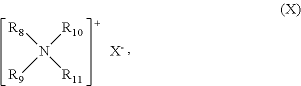
wherein:
groups R₈ to R₁₁, which may be identical or different, represent a linear or branched aliphatic group comprising from 1 to 30 carbon atoms, or an aromatic group, at least one of the groups R₈ to R₁₁ comprising from 8 to 30 carbon atoms;
X⁻ is an anion chosen from halides, phosphates, acetates, lactates, (C₁-C₄)alkyl sulfates, (C₁-C₄)alkylsulfonates, or (C₁-C₄)alkylarylsulfonates;
quaternary ammonium salts of formula (XIII): 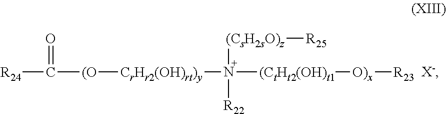
wherein:
R₂₂ is chosen from C₁-C₆ alkyl groups, C₁-C₆ hydroxyalkyl groups or dihydroxyalkyl groups;
R₂₃ is chosen from —C(O)R₂₆; linear or branched, saturated or unsaturated C₁-C₂₂ hydrocarbon-based groups R₂₇; or a hydrogen atom;
R₂₅ is chosen from —C(O)R₂₈; linear or branched, saturated or unsaturated C₁-C₆ hydrocarbon-based groups R₂₉; or a hydrogen atom;
R₂₄, R₂₆ and R₂₈, which may be identical or different, are each chosen from linear or branched, saturated or unsaturated C₇-C₂₁ hydrocarbon-based groups;
r, s and t, which may be identical or different, are integers having values from 2 to 6;
r1 and t1, which may be identical or different, are equal to 0 or 1;
r2+r1=2 r;
t1+t2=2 t;
y is an integer ranging from 1 to 10;
x and z, which may be identical or different, are integers ranging from 0 to 10;
X⁻ is a simple or complex organic anion or mineral anion, with the provisos that the sum x+y+z is from 1 to 15, that when x is 0 then R₂₃ denotes R₂₇, and that when z is 0 then R₂₅ denotes R₂₉; or
mixtures thereof.

34. The composition of claim 17, further comprising: (vii) at least one silicone, wherein the (vii) at least one silicone is chosen from amino silicones.

35. The composition of claim 17, further comprising: (viii) at least one polysaccharide other than cationic polymers, wherein the (viii) at least one polysaccharide other than cationic polymers is chosen from nonionic polysaccharides.

36. A method for dyeing keratin fibers, comprising
applying to the keratin fibers a composition comprising:
(i) at least one compound chosen from N,N-dicarboxymethylglutamic acid, salts thereof, solvates thereof, solvates of the salts thereof, or mixtures thereof;
(ii) at least one coloring agent chosen from oxidation dyes, direct dyes, or mixtures thereof; and
(iii) at least one compound chosen from fatty amines, salts thereof, or mixtures thereof.
